(19) United States
(12) Patent Application Publication
(10) Pub. No.: US 2023/0178215 A1
(43) Pub. Date: Jun. 8, 2023

(54) AUDIO STIMULUS PREDICTION MACHINE LEARNING MODELS

(71) Applicant: UnitedHealth Group Incorporated, Minnetonka, MN (US)

(72) Inventors: Jon Kevin Muse, Thompsons Station, TN (US); Marilyn L. Gordon, Cherry Hill, NJ (US); Garry Choy, Irvine, CA (US); Gregory J. Boss, Saginaw, MI (US)

(21) Appl. No.: 17/643,030

(22) Filed: Dec. 7, 2021

(51) Int. Cl. G16H 20/70

(52) U.S. Cl. CPC G16H 20/70

(57) ABSTRACT

Various embodiments of the present disclosure provide methods, apparatuses, systems, computing devices, computing entities, and/or the like for providing audio stimulation and monitoring patient response information associated therewith. For example, various embodiments provide techniques for generating audio treatment profiles using audio stimulus prediction machine learning models and for use in conjunction with patient monitoring devices.

### BACKGROUND

Comatose patients have been revived with sounds known to be familiar to the patient. As some examples, these stimulating sounds have included the voices of loved ones and music familiar to the patient. However, in most cases, stimulating brain activity in a comatose patient using familiar sounds is highly uncertain and unpredictable. Frequently, it is not possible to accurately determine which sounds were effective or if any sound was the proximate cause of the stimulated brain activity. Moreover, even when brain activity is successfully stimulated, it often takes an extraordinary amount of time and effort for stimulation to be achieved (and for the stimulating sound to be identified, if at all). Compounding these challenges are the enormous costs associated with caring for comatose patients, which increase as a function of the amount of time the patient is in a comatose state.

Accordingly, there is a need in the art for methods of stimulating brain activity in comatose (or partially comatose) patients with greater effectiveness and efficiency. Through applied effort, ingenuity, and innovation, various apparatuses, systems, and methods have been realized for generating an audio treatment profile for a comatose patient and providing effective stimulation to assist with regaining consciousness.

### BRIEF SUMMARY

In general, various embodiments of the present disclosure provide methods, apparatuses, systems, computing devices, computing entities, and/or the like for stimulating brain activity (e.g., in a comatose or partially comatose patient) based at least in part on audio stimulus prediction machine learning models.

In accordance with one aspect, a method is provided. In one embodiment, the method comprises: retrieving, by one or more processors, a plurality of audio stimulus samples; receiving, by the one or more processors, an event data object comprising sensor data describing patient response data of the patient when exposed to the plurality of audio stimulus samples; generating, by the one or more processors and based at least in part on the plurality of audio stimulus samples and the event data object, an audio stimulus map for the patient, wherein the audio stimulus map comprises a mapping of each of the plurality of audio stimulus samples to the patient response data; determining, by the one or more processors, based at least in part on the audio stimulus map and using an audio stimulus prediction machine learning model, an effective subset of the plurality of audio stimulus samples, wherein each audio stimulus sample in the effective subset is associated with a patient response measure that satisfies a patient response measure threshold; identifying, by the one or more processors, one or more audio stimulus patterns of the effective subset; and generating, by the one or more processors, the audio treatment profile based at least in part on the one or more identified audio stimulus patterns of the subset of effective audio stimulus samples, wherein the audio treatment profile may be used to present one or more audio recordings to the patient.

In accordance with another aspect, an apparatus comprising at least one processor and at least one memory including computer program code is provided. In one embodiment, the at least one memory and the computer program code may be configured to, with the processor, cause the apparatus to: retrieve a plurality of audio stimulus samples; receive an event data object comprising sensor data describing patient response data of the patient when exposed to the plurality of audio stimulus samples; generate, based at least in part on the plurality of audio stimulus samples and the event data object, an audio stimulus map for the patient, wherein the audio stimulus map comprises a mapping of each of the plurality of audio stimulus samples to the patient response data; determine, based at least in part on the audio stimulus map and using an audio stimulus prediction machine learning model, an effective subset of the plurality of audio stimulus samples, wherein each audio stimulus sample in the effective subset is associated with a patient response measure that satisfies a patient response measure threshold; identify one or more audio stimulus patterns of the effective subset; and generate the audio treatment profile based at least in part on the one or more identified audio stimulus patterns of the subset of effective audio stimulus samples, wherein the audio treatment profile may be used to present one or more audio recordings to the patient.

In accordance with another aspect, a computer program product is provided. The computer program product may comprise at least one computer-readable storage medium having computer-readable program code portions stored therein, the computer-readable program code portions comprising executable portions configured to retrieve a plurality of audio stimulus samples; receive an event data object comprising sensor data describing patient response data of the patient when exposed to the plurality of audio stimulus samples; generate, based at least in part on the plurality of audio stimulus samples and the event data object, an audio stimulus map for the patient, wherein the audio stimulus map comprises a mapping of each of the plurality of audio stimulus samples to the patient response data; determine, based at least in part on the audio stimulus map and using an audio stimulus prediction machine learning model, an effective subset of the plurality of audio stimulus samples, wherein each audio stimulus sample in the effective subset is associated with a patient response measure that satisfies a patient response measure threshold; identify one or more audio stimulus patterns of the effective subset; and generate the audio treatment profile based at least in part on the one or more identified audio stimulus patterns of the subset of effective audio stimulus samples, wherein the audio treatment profile may be used to present one or more audio recordings to the patient.

### BRIEF DESCRIPTION OF THE DRAWINGS

Having described various embodiments of the invention in general terms, reference will now be made to the accompanying drawings, which are not necessarily drawn to scale, and wherein:

FIG. 1 is an exemplary overview of a system architecture that can be used to practice various embodiments of the present disclosure;

FIG. 2 is an example schematic of an audio stimulus prediction computing entity in accordance with certain embodiments of the present disclosure;

FIG. 3 is an example schematic of an audio stimulation device in accordance with certain embodiments of the present disclosure;

FIG. 4 is an example schematic of an audio stimulus prediction system in accordance with certain embodiments of the present disclosure;

FIG. 5A are example graphs depicting patient response data and audio data, in accordance with certain embodiments of the present disclosure;

FIG. 5B is an operational example depicting an audio stimulus map, in accordance with certain embodiments of the present disclosure;

FIG. 6 is a flowchart diagram illustrating an example process in accordance with certain embodiments of the present disclosure;

FIG. 7 is a flowchart diagram illustrating another example process in accordance with certain embodiments of the present disclosure;

FIG. 8 is a flowchart diagram illustrating yet another example process in accordance with certain embodiments of the present disclosure; and

FIG. 9 is an operational example of generating user interface data in accordance with some embodiments discussed herein.

### DETAILED DESCRIPTION

Various embodiments of the present disclosure now will be described more fully hereinafter with reference to the accompanying drawings, in which some, but not all embodiments are shown. Indeed, various configurations as discussed herein may be embodied in many different forms and should not be construed as limited to the embodiments set forth herein; rather, these embodiments are provided so that this disclosure will satisfy applicable legal requirements. The term “or” (also designated as “I”) is used herein in both the alternative and conjunctive sense, unless otherwise indicated. The terms “illustrative” and “exemplary” are used to be examples with no indication of quality level. Like numbers refer to like elements throughout.

### I. Overview and Technical Advantages

Various embodiments described herein are directed to systems, apparatuses, and methods for stimulating brain activity in comatose (or partially comatose) patients with greater effectiveness and efficiency. In various embodiments, a plurality of audio stimulus samples are played for a patient. In some instances, the audio stimulus examples may be sounds known to be familiar to the patient (e.g., recorded voices of loved ones or music familiar to the patient). Various sensors are then used to monitor the patient's response to being played the audio stimulus samples (e.g., electroencephalography (EEG) sensors for monitoring brain activity).

According to various embodiments, the patient's response to the audio stimulus samples is mapped to generate an audio stimulus sample map (e.g., a map correlating the patient's response to the played audio stimulus samples as a function of time). In order to identify the sounds within the audio stimulus samples that were most effective in stimulating brain activity in the patient, an audio stimulus prediction machine learning model can be used to identify a subset of sounds from the played audio stimulus samples that—based at least in part on the audio stimulus map—were shown to be effective in inducing a patient response (e.g., by identifying patient responses exceeding predefined thresholds and identifying the corresponding sounds that induced those identified patient responses). The audio stimulus patterns of the most effective sounds can then be determined and used to generate an audio treatment profile for the patient.

In various embodiments, audio stimulation prediction machine learning models/techniques may be utilized to analyze the patient's brainwave activity and/or other sensor data in order to identify the most stimulating sounds. In some embodiments, the output of an exemplary audio stimulation prediction machine learning model may be utilized to generate a patient-specific audio treatment profile and/or other forms stimulation for the patient. Therefore, embodiments of the invention provide solutions for the development of effective treatments for comatose patients thereby increasing the efficacy of such treatments and reducing the overall amount of time required to regain consciousness.

Various embodiments are directed to systems, apparatuses, and/or methods for generating an audio treatment profile for use in audio treatment sessions. In some embodiments, a patient monitoring device is configured to monitor patient response during the audio treatment sessions. Accordingly, the audio treatment profile for the patient can be optimized based at least in part on patient response to stimulation over time.

Various embodiments of the present disclosure utilize an EEG-based form of monitoring to determine the most useful sounds delivered to a patient's ears by sensing levels and locations of brain waveform activity generated by the stimulus. This will be enhanced by a more scientific analysis of how specific tones, patterns and environmental sounds invoke even the slightest brainwave activity above life-sustaining brain activity. This can be a fully automated system not requiring human intervention.

Various embodiments of the present disclosure provide a system to analyze brainwaves via EEG or similar technology to detect the most influential sounds activating brain activity. This system of method steps can use headphones or earphones of any type. This will be monitored by traditional EEG embodied in a cap, electrodes, or other advanced EEG monitoring methods. It should be noted that any detection of EEG data above life-sustaining data, whether orderly on not, can be exploited in this system.

In some embodiments, the systems described herein may utilize, real-time 3D imaging to detect and map brainwaves resulting from the varied stimulus. In some embodiments, an example system may incorporate automated muscle stimulus to coincide with sounds. Portions of the brain to a related muscle group where activity is analyzed may be stimulated in sync with automated forms of muscle or physical region touch, pressures or squeezing with electronic compression fabric. This may be presented in many different forms and controlled by Internet of Things (IoT) devices. In some embodiments, an example system may utilize light stimulus to the eyes noting that closed eyelids are not an impacting factor. This could be presented in many different forms controlled by IoT devices and machine learning processing can be employed. In some embodiments, means of introducing tastes, smells, physical stimulation, or changes in the environmental settings may be used to induce brainwave activity in combination with audio stimulus. Additionally, in some embodiments, the techniques described herein may be utilized to provide stimulation to deaf patients. For example, mechanical movement, hot and cold, vibrations, pointy, pulsing or tapping type stimulus can be used to provide stimulation for deaf patients.

Various embodiments of the present disclosure provide a method for dissecting any sound into similar groups of foundational audio stimulus patterns for therapeutic use.

Various embodiments of the present disclosure provide a system to generate and stimulate choice regions of the brain with external audio stimulus.

Various embodiments of the present disclosure provide a system to focus detected brain stimulus on key detected reflexive, biometric, or sensational responses.

Various embodiments of the present disclosure provide a system to suggest specific sound therapy choices for specific comatose patient conditions.

The apparatuses, systems, and methods described herein provide a robust movement monitoring and audio stimulation system. Moreover, various embodiments of the present disclosure provide audio stimulus prediction machine learning models that can make inferences based at least in part on sensor data in a more accurate and computationally efficient manner than state-of-the-art systems. Accordingly, various embodiments of the present disclosure make substantial technical contributions to the field of monitoring devices and substantially improve state-of-the-art systems.

As described herein, various embodiments of the present invention provide practical applications by improving therapeutic stimulation of comatose (or partially comatose) patients with greater effectiveness and efficiency. For example, various embodiments of the present invention generate audio recordings to present to comatose (or partially comatose) patients based at least in part on audio stimulus patterns from those audio stimulus samples that are deemed to be more effective in inducing patient response from comatose (or partially comatose) patients. In doing so, various embodiments of the present improve the effectiveness and efficiency of stimulating comatose (or partially comatose) patients and provide practical solutions for enabling therapeutic stimulation of comatose (or partially comatose) patients.

### II. Definitions of Certain Terms

The term “body” may refer to a person's physical form, and the term may specifically be utilized to refer to a portion of a person's body, including at least a portion of one or more internal and/or external organs of a patient. In general, the terms user, patient, wearer, individual, person, comatose individual, comatose patient, and/or similar terms are used herein interchangeably.

The term “electronically coupled” or “in electronic communication with” may refer to two or more electrical elements (for example, but not limited to, an example processing circuitry, communication module, input/output module memory, plurality of independent foot stimulation sections) and/or electric circuit(s) being connected through wired means (for example but not limited to, conductive wires or traces) and/or wireless means (for example, but not limited to, wireless network, electromagnetic field), such that data and/or information (for example, electronic indications, signals) may be transmitted to and/or received from the electrical elements and/or electric circuit(s) that are electronically coupled.

The term “patient monitoring device” may refer to an article, electrical device and/or EEG-based device that is configured to obtain sensor data describing patient response data (e.g., brainwave information/data). Additionally, in some embodiments, the patient monitoring device may also be configured to provide/deliver stimulation (e.g., audio stimulation, physical stimulation, combinations thereof, and/or the like) to a wearer/patient. In some embodiments, the patient monitoring device may comprise electrodes configured to be worn proximate or adjacent a wearer's head. In various embodiments, the patient monitoring device may be or comprise, for example without limitation, a cap, a hat, headgear, earphones, a jacket, vest, head band, combinations thereof, and/or the like. Additionally, in various embodiments, an example patient monitoring device may comprise at least a power source (e.g., a rechargeable battery), a controller or processor, a wireless communication transceiver and one or more sensors (e.g., electrodes).

The term “sensor data” may refer to one or more data objects describing patient response data to one or more audio stimulus samples. In some embodiments, sensor data may be captured in conjunction with/concurrent with providing audio stimulus (e.g., a plurality of audio stimulus samples) to a patient. In various embodiments, sensor data may comprise physiological information/data, biometric information/data, location information/data, environmental information/data, image/video sensor information/data, and/or the like which may be associated with a particular patient (e.g., a comatose patient). Sensor data may be collected and/or generated by one or more sensors associated with the patient, such as electroencephalography (EEG) sensors, mobile device sensors, patient monitoring device sensors, sensors associated with one or more devices commonly used by the patient, and/or the like. In some embodiments, the sensor data may include image data, inductive probe data, muscle condition data, heart rate data, oxygen saturation data, pulse rate data, body temperature data, breath rate data, perspiration data, blink rate data, blood pressure data, neural activity data, cardiovascular data, pulmonary data, and/or various other types of information/data. In some embodiments, sensor data may be stored in conjunction with a patient profile.

The term “audio stimulus prediction machine learning model” may refer to a data object that describes steps/operations, hyper-parameters, and/or parameters of a machine learning model/algorithm that is configured to generate data needed to infer/generate an audio treatment profile with respect to a person (e.g., a comatose patient). The steps/operations of the audio stimulus prediction machine learning model may lead to performing one or more prediction-based tasks (e.g., providing the audio treatment profile for use in providing audio stimulation to a comatose patient). In some embodiments, the audio stimulus prediction machine learning model may comprise a first sub-model that is configured to generate an audio stimulus map. In some embodiments, the audio stimulus map may be or comprise a table, a graphical illustration and/or map associated with at least a portion of the human body (e.g., the brain). In some embodiments, the audio stimulus prediction machine learning model may comprise a second sub-model that is configured to identify one or more audio stimulus patterns of a subset of effective audio stimulus samples and/or generate an audio treatment profile for the patient. The audio stimulus prediction machine learning model may be trained based at least in part on a ground truth event data object. By way of example, the audio stimulus prediction machine learning model/algorithm may be a neural network, a convolutional neural network (CNN), a recurrent neural network (RNN), and/or the like.

The term “audio treatment profile” may refer to a data object that describes a predictive output of one or more computer-implemented processes, wherein the predictive output describes recommended features of an audio stimulation protocol and/or raw audio data (sounds) that is determined based at least in part on a subset of effective audio stimulus samples (e.g., including ingle, serial, and/or combination sounds) for a particular patient. For instance, the raw audio data may be or comprise voice recording(s), ambient sounds, musical notes, combinations thereof, and/or the like. The example raw audio data may be defined by one or more audio stimulus patterns such as duration, intensity, pitch, frequency and/or the like. Additionally, various instances or portions of the raw audio data may also be associated with one or more parameters such as time of day, physiological parameters, or environmental parameters. For instance, an example sound may be a voice recording that is periodically played at one or more particular times of the day. In some embodiments, determining an audio treatment profile may comprise processing an event data object describing sensor data (e.g., patient response data) associated with a patient. In some embodiments, the audio treatment profile may be an output of the audio stimulus prediction machine learning model. Additionally, in some embodiments, determining the audio treatment profile may comprise identifying a subset of effective audio stimulus samples from a plurality of audio stimulus samples. In some embodiments, the subset of effective audio stimulus samples may be determined based at least in part on a filtering technique or destructive interference technique to distinguish life sustaining waves from brain activity associated with audio stimulus samples. An example destructive interference technique may comprise combining replicated signals to be 180 degrees out of phase to remove unwanted (i.e., life-sustaining) waveforms.

The term “event data object” may refer to a data object storing and/or providing access to information/data that is related to a patient and may describe one or more recorded events/activities associated with the patient. The event data object may comprise sensor data describing patient response data (e.g., neural activity information/data, physiological information/data, image data, body temperature data, and/or the like) of the patient that is captured in conjunction with and/or responsive to delivery of a plurality of audio stimulus samples provided to the patient. Said differently, the event data object may comprise sensor data describing the response of a patient when exposed to the plurality of audio stimulus samples. In some embodiments, an event data object may comprise audio information/data, location information/data, image/video sensor information/data, physiological information/data, biometric information/data, environmental information/data, combinations thereof, and/or the like.

The term “audio stimulus map” may refer to a data object that describes a mapping of each of a plurality of audio stimulus samples to patient response data that is associated with a patient. In some embodiments, each audio stimulus sample may be associated with/mapped to a target location of a patient's brain, a target muscle or muscle group and/or other measurable physical response (e.g., urine output, body temperature, blink rate, and/or the like). By way of example, in an instance in which a particular audio stimulus sample induces neural activity in a particular location of a patient's brain, then the audio stimulus map may describe an association between one or more audio stimulus patterns of the audio stimulus sample and that location of the patient's brain. Similarly, in an instance in which a particular audio stimulus sample induces a physical response/movement in a particular part of the patient's body (e.g., the patient's leg or hand), then the audio stimulus map may describe an association between one or more audio stimulus patterns of the audio stimulus sample and a target location of the patient's body.

### III. Computer Program Products, Methods, and Computing Devices

Embodiments of the present disclosure may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In some embodiments, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In some embodiments, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of a data structure, apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

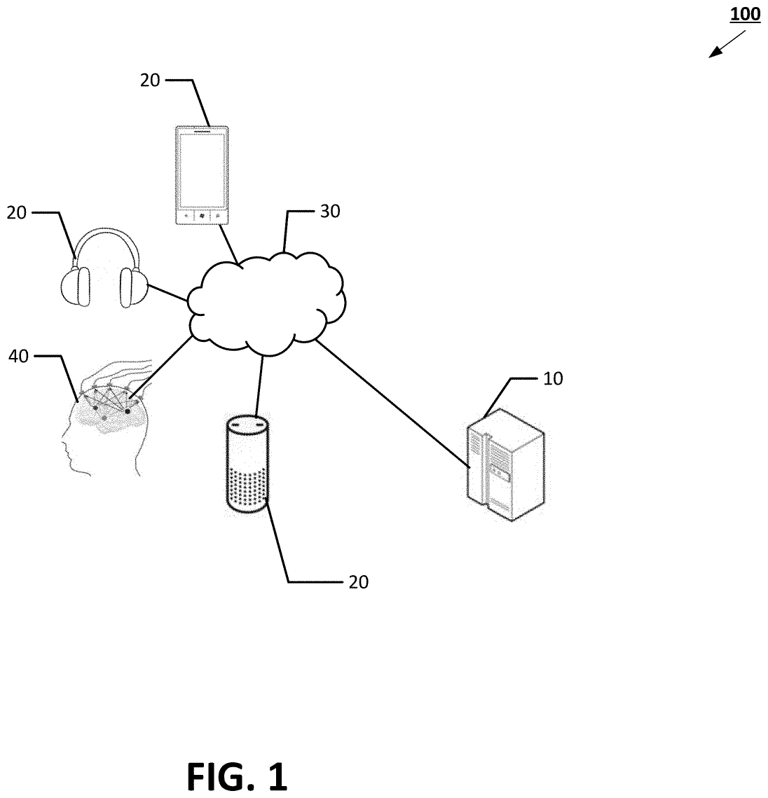

FIG. 1 provides an example system architecture 100 that can be used in conjunction with various embodiments of the present disclosure. As shown in FIG. 1, the system architecture 100 may comprise at least one audio stimulus prediction computing entity 10, an audio stimulation device 20 (e.g., as depicted, one or more electronic devices in communication with the at least one audio stimulus prediction computing entity 10, such as speakers, headphones, portable audio players, and the like), one or more networks 30, one or more patient monitoring devices 40, and/or the like. Each of the components of the system may be in electronic communication with, for example, one another over the same or different wireless or wired networks 30 including, for example, a wired or wireless Personal Area Network (PAN), Local Area Network (LAN), Metropolitan Area Network (MAN), Wide Area Network (WAN), and/or the like. Additionally, while FIG. 1 illustrates certain system devices as separate, standalone devices, the various embodiments are not limited to this particular architecture.

### Exemplary Audio Stimulus Prediction Computing Entity

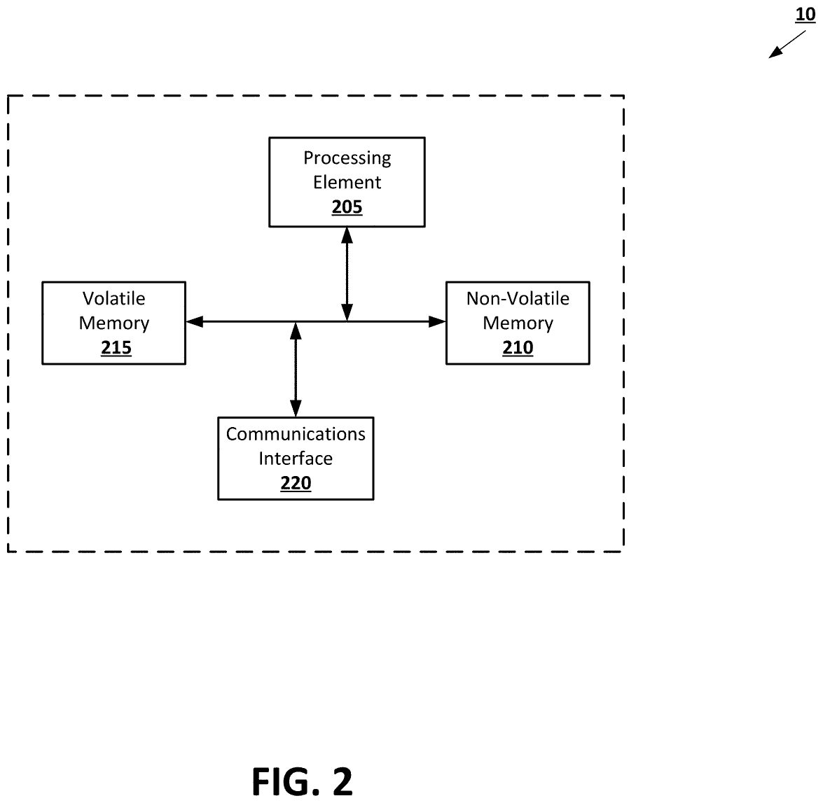

FIG. 2 provides a schematic of an audio stimulus prediction computing entity 10 according to some embodiments of the present disclosure. In general, the terms computing device, entity, device, system, and/or similar words used herein interchangeably may refer to, for example, one or more computers, computing devices, computing entities, desktop computers, mobile phones, tablets, phablets, notebooks, laptops, distributed systems, terminals, servers or server networks, blades, gateways, switches, processing devices, set-top boxes, relays, routers, network access points, base stations, the like, and/or any combination of devices adapted to perform the functions, operations, and/or processes described herein. Such functions, operations, and/or processes may include, for example, transmitting, receiving, operating on, processing, displaying, storing, determining, generating/creating, monitoring, evaluating, comparing, and/or similar terms used herein interchangeably. In some embodiments, these functions, operations, and/or processes can be performed on data, content, information, and/or similar terms used herein interchangeably.

As indicated, in some embodiments, the audio stimulus prediction computing entity 10 may also include one or more network and/or communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in some embodiments, the audio stimulus prediction computing entity 10 may include or be in communication with one or more processing elements 205 (also referred to as processors, processing circuitry, and/or similar terms used herein interchangeably) that communicate with other elements within the audio stimulus prediction computing entity 10 via a bus, for example. As will be understood, the processing element 205 may be embodied in a number of different ways. For example, the processing element 205 may be embodied as one or more complex programmable logic devices (CPLDs), microprocessors, multi-core processors, coprocessing devices, application-specific instruction-set processors (ASIPs), and/or controllers. Further, the processing element 205 may be embodied as one or more other processing devices or circuitry. The term circuitry may refer to an entire hardware embodiment or a combination of hardware and computer program products. Thus, the processing element 205 may be embodied as integrated circuits, application specific integrated circuits (ASICs), field-programmable gate arrays (FPGAs), programmable logic arrays (PLAs), hardware accelerators, other circuitry, and/or the like. As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

In some embodiments, the audio stimulus prediction computing entity 10 may further include or be in communication with non-volatile media (also referred to as non-volatile storage, memory, memory storage, memory circuitry and/or similar terms used herein interchangeably). In some embodiments, the non-volatile storage or memory may include one or more non-volatile storage or memory media 210 as described above, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or memory media may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably may refer to a structured collection of records or information/data that is stored in a computer-readable storage medium, such as via a relational database, hierarchical database, and/or network database.

In some embodiments, the audio stimulus prediction computing entity 10 may further include or be in communication with volatile media (also referred to as volatile storage, memory, memory storage, memory circuitry, and/or similar terms used herein interchangeably). In some embodiments, the volatile storage or memory may also include one or more volatile storage or memory media 215 as described above, such as RAM, DRAM, SRAM, FPM DRAM, EDO DRAM, SDRAM, DDR SDRAM, DDR2 SDRAM, DDR3 SDRAM, RDRAM, RIMM, SIMM, VRAM, cache memory, register memory, and/or the like. As will be recognized, the volatile storage or memory media may be used to store at least portions of the databases, database instances, database management system entities, data, applications, programs, program modules, scripts, source code, object code, byte code, compiled code, interpreted code, machine code, executable instructions, and/or the like being executed by, for example, the processing element 205. Thus, the databases, database instances, database management system entities, data, applications, programs, program modules, scripts, source code, object code, byte code, compiled code, interpreted code, machine code, executable instructions, and/or the like may be used to control certain aspects of the operation of the audio stimulus prediction computing entity 10 with the assistance of the processing element 205 and the operating system.

As indicated, in some embodiments, the audio stimulus prediction computing entity 10 may also include one or more network and/or communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, audio stimulus prediction computing entity 10 may be configured to communicate via wireless external communication networks using any of a variety of protocols, such as general packet radio service (GPRS), Universal Mobile Telecommunications System (UMTS), Code Division Multiple Access 200 (CDMA200), CDMA200 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), IR protocols, NFC protocols, RFID protocols, IR protocols, ZigBee protocols, Z-Wave protocols, 6LoWPAN protocols, Wibree, Bluetooth protocols, wireless universal serial bus (USB) protocols, and/or any other wireless protocol. The audio stimulus prediction computing entity 10 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

As will be appreciated, one or more of the audio stimulus prediction computing entity's components may be located remotely from one another, such as in a distributed system. Furthermore, one or more of the components may be aggregated and additional components performing functions described herein may be included in the audio stimulus prediction computing entity 10. Thus, the audio stimulus prediction computing entity 10 can be adapted to accommodate a variety of needs and circumstances, such as including various components described with regard to a mobile application executing on the audio stimulation device 20, including various input/output interfaces.

### Exemplary Audio Stimulation Device

The audio stimulation device 20 may be in communication with the audio stimulus prediction computing entity 10 and the patient monitoring device 40. The audio stimulation device 20 may obtain and provide (e.g., transmit/send) data objects describing raw data (e.g., sensor data such as physiological data associated with the patient) obtained by one or more additional sensors or sensing devices, captured by another computing entity or device and/or provided by another computing entity. The audio stimulation device 20 may be configured to provide (e.g., transmit, send) data objects describing at least a portion of the sensor data to the audio stimulus prediction computing entity 10. Additionally, in various embodiments, a remote computing entity may provide data objects describing patient information/data to the audio stimulus prediction computing entity 10. In some embodiments, an operator of the patient monitoring device 40 may operate the patient monitoring device 40 via the display 316 or keypad 318 of the audio stimulation device 20.

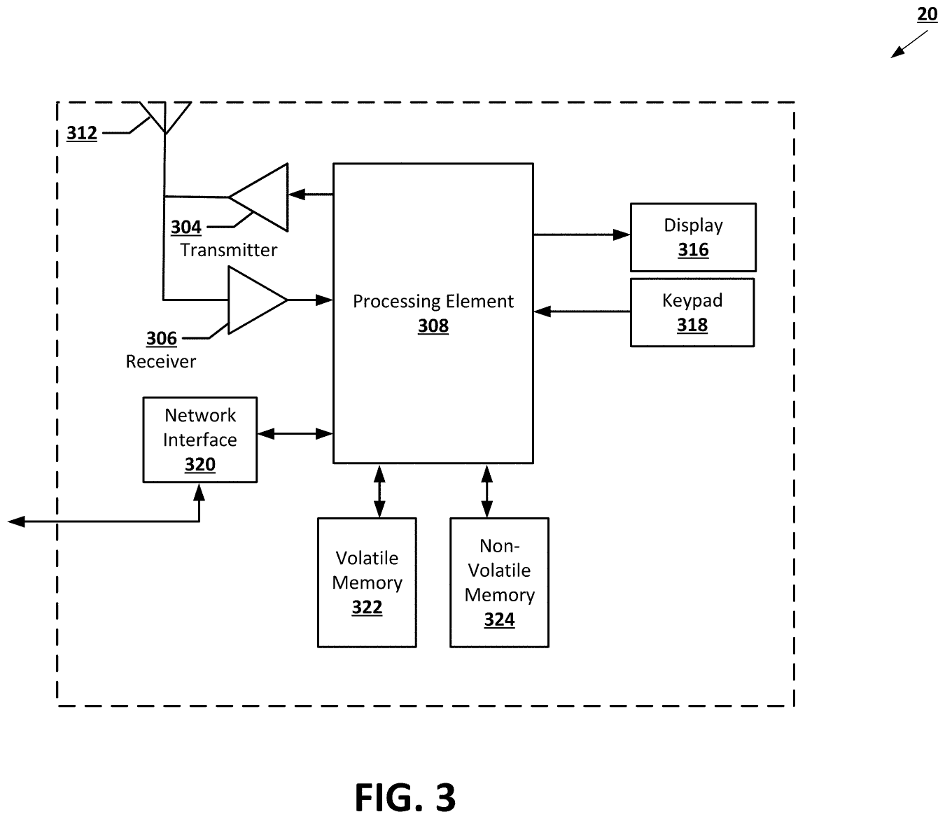

FIG. 3 provides an illustrative schematic representative of audio stimulation device 20 that can be used in conjunction with embodiments of the present disclosure. In various embodiments, the audio stimulation device 20 may be or comprise one or more mobile devices and/or electronic devices. For example, an audio stimulation device 20 may be embodied as or operate in conjunction with a mobile device, headphones, speaker, combinations thereof, or any other computing device configured to provide audio stimulation to the patient and/or obtain sensor data from the patient. In some embodiments, the audio stimulation device 20 may be in electronic communication with or in close proximity to a patient monitoring device 40 worn by the patient, such that close-range wireless communication technologies may be utilized for communicating between a controller of a patient monitoring device 40 and the audio stimulation device 20.

As shown in FIG. 3, an audio stimulation device 20 can include an antenna 312, a transmitter 304 (e.g., radio), a receiver 306 (e.g., radio), and a processing element 308 that provides signals to and receives signals from the transmitter 304 and receiver 306, respectively. The signals provided to and received from the transmitter 304 and the receiver 306, respectively, may include signaling information/data in accordance with an air interface standard of applicable wireless systems to communicate with various devices, such as an audio stimulus prediction computing entity 10, another audio stimulation device 20, computing device, and/or the like. In an example embodiment, the transmitter 304 and/or receiver 306 are configured to communicate via one or more SRC protocols. For example, the transmitter 304 and/or receiver 306 may be configured to transmit and/or receive information/data, transmissions, and/or the like of at least one of Bluetooth protocols, low energy Bluetooth protocols, NFC protocols, RFID protocols, IR protocols, Wi-Fi protocols, ZigBee protocols, Z-Wave protocols, 6LoWPAN protocols, and/or other short range communication protocol. In various embodiments, the antenna 312, transmitter 304, and receiver 306 may be configured to communicate via one or more long range protocols, such as GPRS, UMTS, CDMA200, 1xRTT, WCDMA, GSM, EDGE, TD-SCDMA, LTE, E-UTRAN, EVDO, HSPA, HSDPA, Wi-Fi, Wi-Fi Direct, WiMAX, and/or the like. The audio stimulation device 20 may also include one or more network and/or communications interfaces 320 for communicating with various computing entities, such as by communicating data, content, information, and/or similar terms used herein interchangeably that can be transmitted, received, operated on, processed, displayed, stored, and/or the like.

In this regard, the audio stimulation device 20 may be capable of operating with one or more air interface standards, communication protocols, modulation types, and access types. More particularly, the audio stimulation device 20 may operate in accordance with any number of wireless communication standards and protocols. In a particular embodiment, the audio stimulation device 20 may operate in accordance with multiple wireless communication standards and protocols, such as GPRS, UMTS, CDMA200, 1xRTT, WCDMA, TD-SCDMA, LTE, E-UTRAN, EVDO, HSPA, HSDPA, Wi-Fi, WiMAX, UWB, IR protocols, Bluetooth protocols, USB protocols, and/or any other wireless protocol.

Via these communication standards and protocols, the audio stimulation device 20 can communicate with various other devices using concepts such as Unstructured Supplementary Service information/data (USSD), Short Message Service (SMS), Multimedia Messaging Service (MMS), Dual-Tone Multi-Frequency Signaling (DTMF), and/or Subscriber Identity Module Dialer (SIM dialer). The audio stimulation device 20 can also download changes, add-ons, and updates, for instance, to its firmware, software (e.g., including executable instructions, applications, program modules), and operating system.

According to some embodiments, the audio stimulation device 20 may include location determining aspects, devices, modules, functionalities, and/or similar words used herein interchangeably to acquire location information/data regularly, continuously, or in response to certain triggers. For example, the audio stimulation device 20 may include outdoor positioning aspects, such as a location module adapted to acquire, for example, latitude, longitude, altitude, geocode, course, direction, heading, speed, UTC, date, and/or various other information/data. In some embodiments, the location module can acquire information/data, sometimes known as ephemeris information/data, by identifying the number of satellites in view and the relative positions of those satellites. The satellites may be a variety of different satellites, including LEO satellite systems, DOD satellite systems, the European Union Galileo positioning systems, the Chinese Compass navigation systems, Indian Regional Navigational satellite systems, and/or the like. Alternatively, the location information/data may be determined by triangulating the apparatus's 30 position in connection with a variety of other systems, including cellular towers, Wi-Fi access points, and/or the like. Similarly, the audio stimulation device 20 may include indoor positioning aspects, such as a location module adapted to acquire, for example, latitude, longitude, altitude, geocode, course, direction, heading, speed, time, date, and/or various other information/data. Some of the indoor aspects may use various position or location technologies including RFID tags, indoor beacons or transmitters, Wi-Fi access points, cellular towers, nearby computing entities (e.g., smartphones, laptops) and/or the like. For instance, such technologies may include iBeacons, Gimbal proximity beacons, BLE transmitters, NFC transmitters, and/or the like. These indoor positioning aspects can be used in a variety of settings to determine the location of someone or something to within inches or centimeters.

The audio stimulation device 20 may also comprise a user interface device comprising one or more user input/output interfaces (e.g., a display 316 and/or speaker/speaker driver coupled to a processing element 308 and a touch interface, keyboard, mouse, and/or microphone coupled to a processing element 308). For example, the patient interface may be configured to provide a mobile application, browser, interactive user interface, dashboard, webpage, and/or similar words used herein interchangeably executing on and/or accessible via the audio stimulation device 20 to cause the display or audible presentation of information/data and for user interaction therewith via one or more user input interfaces. Moreover, the patient interface can comprise or be in communication with any of a number of devices allowing the audio stimulation device 20 to receive information/data, such as a keypad 318 (hard or soft), a touch display, voice/speech or motion interfaces, scanners, readers, or other input device. In embodiments including a keypad 318, the keypad 318 can include (or cause display of) the conventional numeric (0-9) and related keys (#, *), and other keys used for operating the audio stimulation device 20 and may include a full set of alphabetic keys or set of keys that may be activated to provide a full set of alphanumeric keys. In addition to providing input, the patient input interface can be used, for example, to activate or deactivate certain functions, such as screen savers and/or sleep modes. Through such inputs the audio stimulation device 20 can capture, collect, store information/data, user interaction/input, and/or the like.

The audio stimulation device 20 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory can store databases, database instances, database management system entities, information/data, applications, programs, program modules, scripts, source code, object code, byte code, compiled code, interpreted code, machine code, executable instructions, and/or the like to implement the functions of the audio stimulation device 20.

In various embodiments, the audio stimulation device 20 may be embodied as an artificial intelligence (AI) computing entity, such as an Amazon Echo, Amazon Echo Dot, Amazon Show, Google Home, and/or the like. Accordingly, the audio stimulation device 20 may be configured to provide and/or receive information/data from an operator via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms (in some examples, upon the occurrence of a predefined trigger event or in accordance with a predetermined schedule).

### Exemplary Audio Stimulus Prediction System

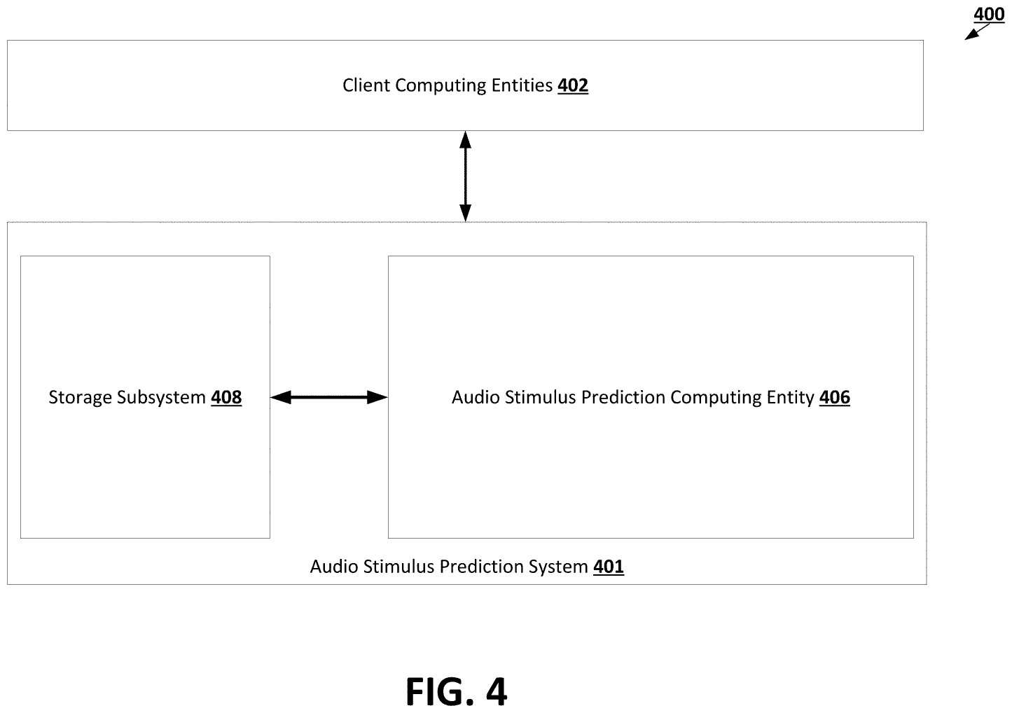

FIG. 4 is a schematic diagram of an example system architecture 400 for generating an audio treatment profile that can be used to perform one or more prediction-based tasks. The architecture 400 includes an audio stimulus prediction system 401 that is configured to receive data from the client computing entities 402, process the data to generate predictive outputs (e.g., audio treatment profile data objects), and provide the outputs to the client computing entities 402 (e.g., for generating user interface data and/or dynamically updating a user interface). In some embodiments, audio stimulus prediction system 401 may communicate with at least one of the client computing entities 402 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The audio stimulus prediction system 401 may include an audio stimulus prediction computing entity 406 and a storage subsystem 408. The audio stimulus prediction computing entity 406 may be configured to receive queries, requests and/or data from client computing entities 402, process the queries, requests and/or data to generate predictive outputs, and provide (e.g., transmit, send, and/or the like) the predictive outputs to the client computing entities 402.

The client computing entities 402 may be configured to transmit requests to the audio stimulus prediction computing entity 406 in response to queries. Responsive to receiving the predictive outputs, the client computing entities 402 may generate user interface data and may provide (e.g., transmit, send and/or the like) user interface data for presentation by user computing entities.

The storage subsystem 408 may be configured to store at least a portion of the data utilized by the audio stimulus prediction computing entity 406 to perform audio stimulus prediction operations and tasks. The storage subsystem 408 may be configured to store at least a portion of operational data and/or operational configuration data including operational instructions and parameters utilized by the audio stimulus prediction computing entity 406 to perform audio stimulus prediction operations/tasks in response to requests. The storage subsystem 408 may include one or more storage units, such as multiple distributed storage units that are connected through a computer network. Each storage unit in the storage subsystem 408 may store at least one of one or more data assets and/or one or more data about the computed properties of one or more data assets. Moreover, each storage unit in the storage subsystem 408 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Patient Monitoring Device

In various embodiments, the patient monitoring device 40 is configured to obtain sensor data (e.g., patient response data such as brainwave information/data) and/or provide audio stimulation and/or other forms of stimulation to a comatose patient. As noted above, the patient monitoring device 40 may be or comprise an article configured to be worn proximate or adjacent a wearer's head. For example, the patient monitoring device 40 may comprise a plurality of electrodes configured to obtain sensor data describing patient response data. Additionally and/or alternatively, in some embodiments, the patient monitoring device 40 may further comprise or be in electronic communication with one or more other devices that are configured to provide audio stimulation and/or other forms of stimulation such as earphones, a speaker, a mobile device combinations thereof, and/or the like. In some embodiments, the patient monitoring device 40 may comprise or be in electronic communication with one or more additional sensors such as image sensors, physiological sensors, temperature sensors, biometric sensors, combinations thereof, and/or the like. For example, the one or more additional sensors may be configured to capture patient response data (e.g., brainwave information/data, physiological information/data), environmental data/information (e.g., ambient temperature, light level information), combinations thereof, and/or the like. In some embodiments, patient response data may comprise taste or smell sensations, goose bumps, flushing, variations in known benign conditions or involuntary sexual responses. In some embodiments, the patient monitoring device 40 may comprise a wearable magnetic resonance imaging (MRI) device.

In certain embodiments, at least one sensor of the patient monitoring device 40 enables receiving and/or capturing raw sensor information/data (e.g., regularly, continuously, and/or in response to certain triggers). In some embodiments, the patient monitoring device 40 and/or other electronic device(s) may comprise microelectromechanical (MEMS) components, biological and chemical sensing components, electrocardiogram (ECG) components, electromyogram (EMG) components, EEG-based neural sensing components, optical sensing components, electrical sensing components, sound components, vibration sensing components, accelerometer(s), pressure sensor(s) and/or the like. In certain embodiments, the at least one sensor may comprise a plurality of sensors of various sensor types to capture multiple data types. In certain embodiments, sensor data from one or more sensors may be analyzed (e.g., locally by the controller of the patient monitoring device 40 or via the audio stimulus prediction computing entity 10) to generate an audio treatment profile. Through such components, various types of physiological information/data can be captured—such as heart rate information/data, oxygen saturation information/data, body temperature information/data, breath rate information/data, perspiration information/data, neural information/data, cardiovascular sounds information/data, and/or various other types of information/data. The one or more sensors of the patient monitoring device 40 may be in electronic communication with the controller of the patient monitoring device 40 such that they can exchange information/data (e.g., receive and transmit data) with the patient monitoring device 40. Additionally, sensor data may be collected and/or generated by one or more sensors associated with the patient, such as patient monitoring device sensors (e.g., a smartwatch), sensors associated with one or more devices commonly used by the patient (e.g., a glucose monitoring device), IoT devices in the patient's environment, and/or the like.

In some embodiments, the controller of the patient monitoring device 40 (e.g., which may comprise a computing device, one or more computer processors, or the like) may include a wireless communication transceiver and/or the like. In various embodiments, the controller of the patient monitoring device 40 may comprise components similar or identical to the audio stimulation device 20 depicted in FIG. 3. The controller may be integrated into or attached to any surface of the patient monitoring device 40 and may be in wired or wireless communication with various elements (e.g., one or more sensors described above, and/or the like) of the patient monitoring device 40, and a power source of the patient monitoring device. Accordingly, the controller of the patient monitoring device 40 may be configured to (e.g., alone or together with the audio stimulus prediction computing entity 10) provide appropriate signals to elements of the patient monitoring device 40 and/or other electronic devices in order to provide audio stimulation. In some embodiments, the controller may be in wireless communication with, but be physically distinct from, the patient monitoring device 40 (e.g., via short-range wireless communication, such as Bluetooth communication, via long-range wireless communication, and/or the like), which may encompass a wireless receiver, thereby enabling appropriate signals to be passed to the patient monitoring device 40 as discussed herein. In certain embodiments, the controller may comprise an input/output interface system comprising one or more user input/output interfaces (e.g., a button, a display, and a touch interface, and/or a microphone coupled to a processing element and/or controller). For example, the patient interface may be configured to cause display of or present audible presentation of information/data and for interaction therewith via one or more user input interfaces. The controller may store instructions/parameters required for various operations by the patient monitoring device 40.

As discussed herein, the controller may comprise one or more control elements for transmitting a control signal to control (e.g., adjust or modify) various operations and operational parameters of the patient monitoring device 40. For example, an operator (e.g., clinician) may control (e.g., override) the patient monitoring device 40, for example in order to adjust features of or stop operations of the patient monitoring device 40.

### V. Exemplary System Operations

As described below, the apparatuses, systems, and methods described herein provide a robust system for providing generating an audio treatment profile and providing audio stimulation in the form of sound waves (e.g., via a patient monitoring device and/or other electronic device). Moreover, various embodiments of the present disclosure provide audio stimulus prediction machine learning models that can make inferences based at least in part on sensory data in order to perform prediction-based tasks in a more computationally efficient manner than state-of-the-art systems. Accordingly, various embodiments of the present disclosure make substantial technical contributions to the field of monitoring devices and substantially improve state-of-the-art systems.

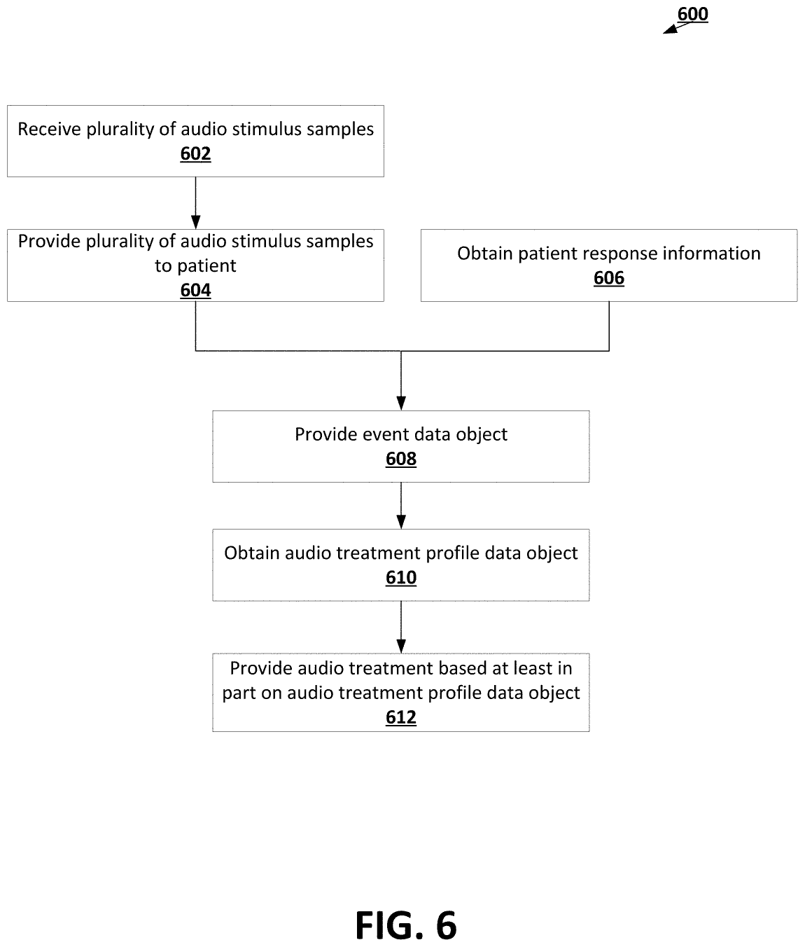

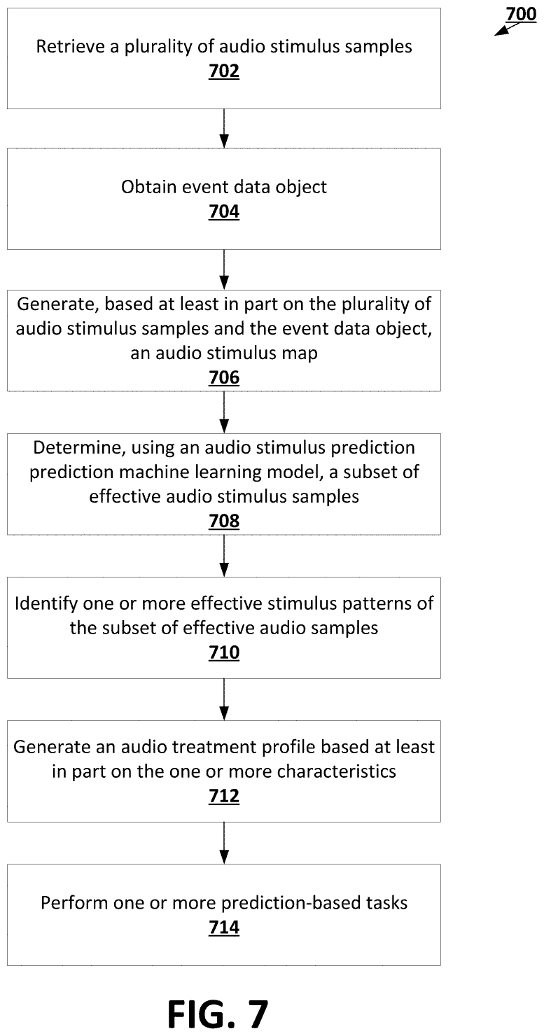

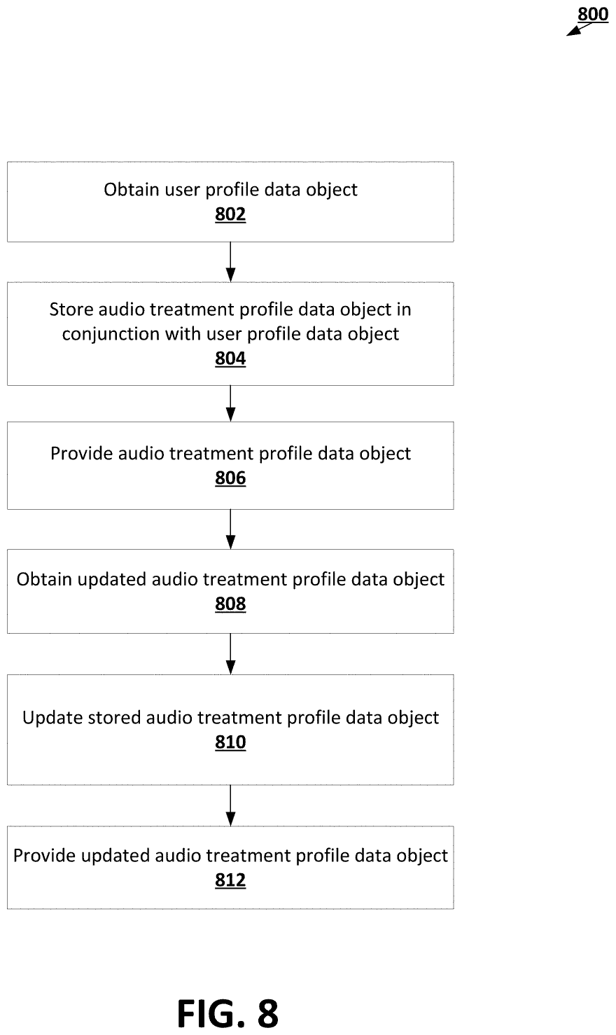

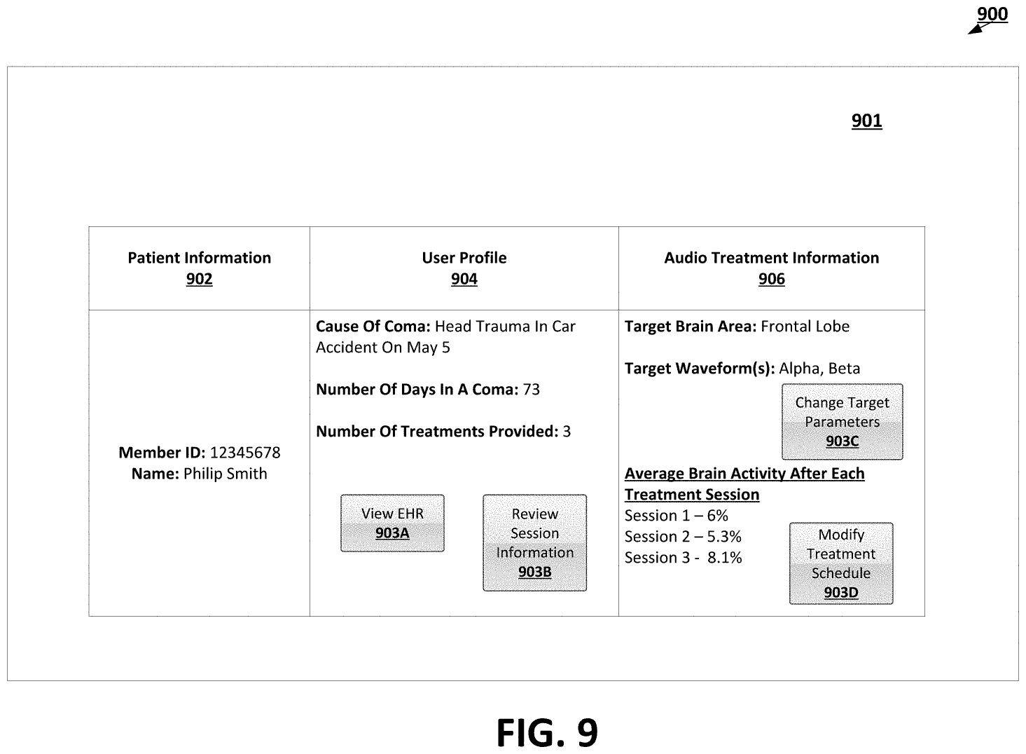

FIG. 6, FIG. 7, and FIG. 8 are flowcharts illustrating example steps, processes, procedures, and/or operations; FIG. 9 is an operational example of generating user interface data in accordance with some embodiments discussed herein. Although the following exemplary operations are described as being performed by one of the patient monitoring device 40 (e.g., via the controller), the audio stimulus prediction computing entity 10, or the audio stimulation device 20, it should be understood that in various embodiments, the operations can be interchangeably performed by other components within the system architecture 100.

Various embodiments may be configured to utilize one or more patient profiles (e.g., a patient-specific audio treatment profile) to facilitate operations of the patient monitoring device 40 and/or other electronic device (e.g., earphones, speaker or the like). The patient-specific audio treatment profile may comprise data indicative of features of the patient (e.g., data indicative of the patient's age, gender, medical conditions, and/or the like, which may be obtained from electronic medical record (EMR) data stored in a data storage area and associated with the patient), as well as data indicative of functional results of operations of the patient monitoring device (e.g., data relating to historical patient response data) determined based at least in part on the operation of the sensors of the patient monitoring device 40 and/or additional sensors. Accordingly, the audio stimulus prediction computing entity 10 may be configured to obtain (e.g., receive) and process data objects describing raw data (sensor data, physiological data, patient profile information/data, and/or the like) associated with a patient in order to generate an audio treatment profile for the patient. An example audio treatment profile may comprise raw audio data that is determined based at least in part on a subset of effective audio stimulus samples for a particular patient. For example, raw audio data may comprise a plurality of sound waves that each comprise a wavelength oscillating at a given frequency for a duration of time. Each sound wave (or instance/portion of raw audio data) may be defined by one or more audio stimulus patterns such as duration, intensity, pitch, frequency, amplitude, and/or the like. For instance, an example instance of raw audio data may comprise a voice recording, ambient sounds, musical notes, combinations thereof, and/or the like.

An audio treatment profile data object may be stored in conjunction with or otherwise associated with a patient profile data object. In some embodiments, an operator (e.g., a clinician or the wearer) interfacing with the audio stimulus prediction computing entity 10 may modify the audio treatment profile data object and/or select instances of raw audio data associated with the audio treatment profile data object. The audio stimulus prediction computing entity 10 may be configured to store and/or in turn provide (e.g., send, transmit) the audio treatment profile data object to a patient monitoring device 40 and/or other electronic device. The audio stimulus prediction computing entity 10 may be configured to obtain (e.g., receive, request) and process a data object describing raw sensor data collected by sensors of the patient monitoring device 40 (e.g., electrodes) and/or other sensors and sensing devices associated with the patient in order to update the audio treatment profile data object and the stored stimulation protocols for the patient. The audio stimulus prediction computing entity 10 may be configured to process (periodically or in response to receiving particular data) additional data/information associated with the patient in order to update (e.g., adjust, change) the audio treatment profile data object for the patient. The audio stimulus prediction computing entity 10 may periodically provide (e.g., send, transmit) an up-to-date audio treatment profile to the patient monitoring device 40 or other electronic device. The audio stimulus prediction computing entity 10 may generate a user interface data object corresponding with the audio treatment profile data object and provide (e.g., transmit, send, and/or the like) the patient interface data object to one or more computing entities (e.g., other computing entities operated by the clinician, and/or the like) for presentation by the noted computing entities.

### Exemplary Techniques for Generating an Audio Treatment Profile

In various embodiments, an example audio stimulus prediction computing entity 10 may be configured to generate an audio treatment profile. In various embodiments, a patient monitoring device 40 and/or other electronic device may be configured to store an audio treatment profile comprising raw audio data (e.g., voice recording(s), ambient sounds, musical notes, combinations thereof, and/or the like). The audio treatment profile may be utilized to provide stimulation to a comatose patient.

Referring now to FIG. 7, a flowchart diagram illustrating an example process 700 for generating an audio treatment profile by an audio stimulus prediction computing entity 10 in accordance with some embodiments of the present disclosure is provided.

In some embodiments, beginning at step/operation 702, the audio stimulus prediction computing entity 10 retrieves a plurality of audio stimulus samples. As noted above, the audio stimulus prediction computing entity 10 may obtain the plurality of audio stimulus samples from one or more databases or sound libraries and/or the audio stimulation device 20. Accordingly, in some embodiments, the audio stimulus prediction computing entity 10 may retrieve and provide at least a portion of the plurality of audio stimulus samples required for performing analyses and/or providing stimulation to the patient as part of an audio treatment protocol.

Subsequent to step/operation 702, the process 700 proceeds to step/operation 704. At step/operation 704, the audio stimulus prediction computing entity 10 obtains the event data object (e.g., from the audio stimulation device 20) describing the plurality of audio stimulus samples (that were provided to the patient) and/or patient response data associated therewith.

Subsequent to step/operation 704, the process 700 proceeds to step/operation 706. At step/operation 706, the audio stimulus prediction computing entity 10 generates, based at least in part on the plurality of audio stimulus samples and the event data object, an audio stimulus map for the patient. Accordingly, in various embodiments, data generated and/or collected as a part of providing a plurality of audio stimulus samples may be utilized to generate the audio stimulus map for the patient. By monitoring the patient's responses to each audio stimulus sample as well as associated parameters (e.g., physiological and/or environmental parameters), the audio stimulus prediction computing entity 10 can associate particular audio stimulus samples with patient response data.

An audio stimulus map may describe a mapping of each of a plurality of audio stimulus samples to patient response data that is associated with a patient. In some embodiments, the audio stimulus map may comprise patient response data (e.g., as measured via one or more sensors) mapped to one or more sound waves as a function of time. Said differently, for a given point in time, the audio stimulus map may describe one or more sound waves and a patient's response to the one or more sound waves. Each audio stimulus sample may be associated with/mapped to a target location of a patient's brain, a target muscle group and/or other measurable physical responses (e.g., urine output, body temperature, blink rate, and/or the like). By way of example, in an instance in which a particular audio stimulus sample induces EEG/neural activity in a particular location of a patient's brain, then the audio stimulus map may describe an association between the audio stimulus sample with that location of the patient's brain. Similarly, in an instance in which a particular audio stimulus sample induces a physical response/movement in a particular part of the patient's body (e.g., the patient's leg or hand), then the audio stimulus map may describe an association between the audio stimulus sample and a target location of the patient's body. In some embodiments, the audio stimulus map may be embodied as a table, a graphical illustration or map associated with at least a portion of the human body (e.g., the brain), and/or the like.

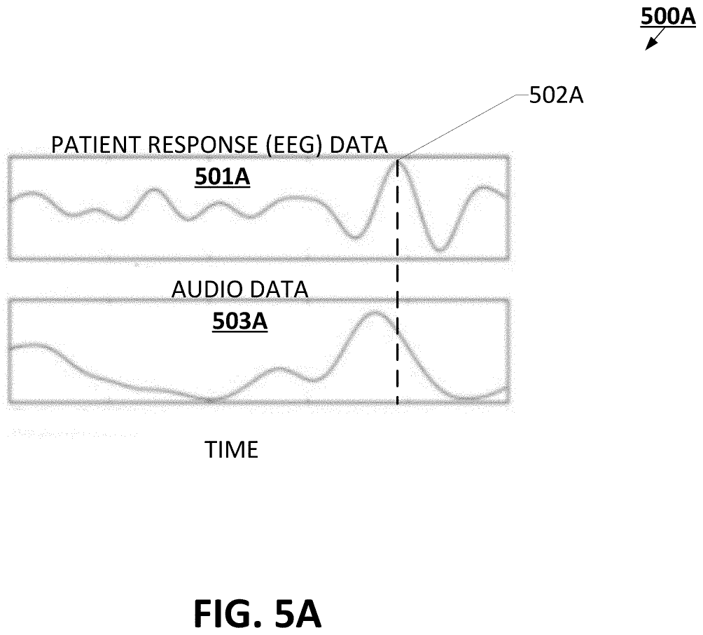

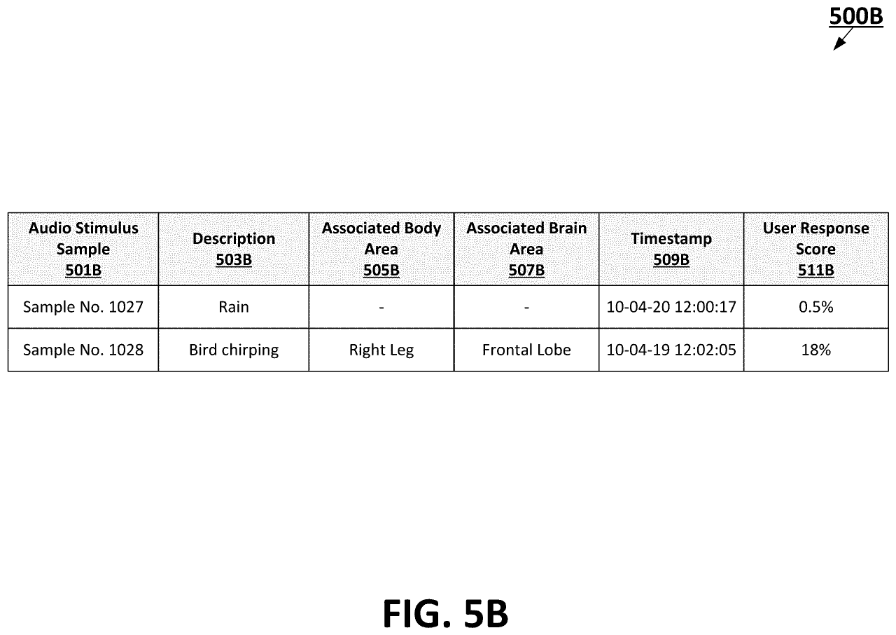

Referring now to FIG. 5A, example graphs 500A depicting patient response data 501A and audio data 503A in accordance with certain embodiments of the present disclosure are provided. As depicted in FIG. 5A, the x-axis represents a plurality of instances in time. As depicted, the y-axis in the graph depicting patient response data 501A represents a plurality of measurements (e.g., in microvolts) associated with EEG activity. As further depicted, the y-axis in the graph depicting audio data 503A represents a waveform of an audio stimulus sample (e.g., a frequency domain representation). As illustrated, a significant peak 502A occurring at a particular instance in time in the patient response data 501A corresponds with a similar peak near the same instance in time shown in the audio data 503A. Accordingly, patient response data 501A may be correlated with audio data 503A by identifying peaks at or around particular instances in time. In some embodiments, multiples sources of audio sounds (e.g., represented by different waveforms) may be analyzed simultaneously and/or sequentially. By way of example, the audio data 503A may comprise a plurality of waveforms associated with different sounds (e.g., background noise, street traffic, speech, music, and the like).

Referring now to FIG. 5B, an operational example depicting an audio stimulus map 500B in accordance with certain embodiments of the present disclosure is provided. As depicted in FIG. 5B, the example audio stimulus map 500B comprises a table mapping each audio stimulus sample of a plurality of audio stimulus samples 501B to patient response data. As illustrated, each audio stimulus sample 501B is associated with a description 503B, an associated body area 505B, an associated brain area 507B, timestamp data 509B, and patient response data. In various embodiments, the patient response data may comprise raw data/values. In some embodiments, as depicted, the patient response data may be a patient response score 511B. As such, in some embodiments, the patient response score 511B may be an inferred determination relating to the patient's level of response to the particular audio stimulus sample 501B.

In some embodiments, generating the audio stimulus map comprises identifying baseline life-sustaining wave audio stimulus patterns in the brainwave information/data for the patient. For example, the audio stimulus prediction computing entity 10 may distinguish life-sustaining waves from brain activity associated with audio stimulus samples using a filtering technique or destructive interference technique. The example destructive interference technique may comprise combining replicated signals to be 180 degrees out of phase to remove unwanted waveforms.

In some embodiments, the audio stimulus prediction computing entity 10 may utilize an audio stimulus prediction machine learning model to generate the audio stimulus map. In various embodiments, the audio stimulus prediction computing entity may be a data object that describes steps/operations, hyper-parameters, and/or parameters of a machine learning model/algorithm that is configured to generate data needed to infer/generate an audio treatment profile with respect to an individual (e.g., a comatose patient). The steps/operations of the audio stimulus prediction machine learning model may lead to performing one or more prediction-based tasks (e.g., providing the audio treatment profile for use in providing audio stimulation to a comatose patient). The audio stimulus prediction machine learning model may comprise a first sub-model that is configured to generate the audio stimulus map and a second sub-model that is configured to identify one or more audio stimulus patterns of a subset of effective audio stimulus samples and/or generate the audio treatment profile for the patient. The audio stimulus prediction machine learning model may be trained based at least in part on a ground truth event data object. In some embodiments, the audio stimulus prediction machine learning model/algorithm may be a neural network, a convolutional neural network (CNN), a recurrent neural network (RNN), and/or the like.

Subsequent to step/operation 706, the process 700 proceeds to step/operation 708. At step/operation 708, audio stimulus prediction computing entity 10 determines, using an audio stimulus prediction machine learning model, a subset of effective audio stimulus samples from the plurality of audio stimulus samples (also referred to herein as an “effective subset of the plurality of audio stimulus samples”). In some embodiments, the audio stimulus prediction computing entity 10 may detect spikes in brainwave information/data coinciding with certain audio stimulus samples provided to the patient. The audio stimulus prediction computing entity 10 may tag/label particular entries of audio stimulus samples based at least in part on an inferred effectiveness measure and/or may order the audio stimulus samples from most effective to least effective. In some embodiments, the audio stimulus prediction computing entity 10 determines the subset of effective samples by applying frequency spectrum analysis to each of the plurality of audio stimulus samples. In various embodiments, frequency spectrum analysis may be performed in real-time or only on patient response data sets that satisfy a patient response threshold. In some examples, the patient response threshold may be a value such as an EEG-related value (e.g., a percentage value or a number between 0 and 1), where an above-threshold value indicates that the audio stimulus sample is effective and/or likely to be effective. In some embodiments, audio stimulus prediction computing entity 10 may identify frequency spectrums of each successful audio stimulus sample and apply like singular sounds to possibly pinpoint the most stimulating sound/feature. By way of example, the audio stimulus prediction computing entity 10 may apply a plurality of audio stimulus samples comprising piano notes in the same spectrum as broadly detected sounds. Each of these may be tagged, ordered and/or stored (e.g., ordered from most effective to least effective). In some embodiments, a listing of audio stimulus samples may be ordered according to identified frequency range (e.g., broad, or highest to lowest). Additionally and/or alternatively, audio stimulus prediction computing entity 10 may subdivide sound into the sections, such as divided according to volume intensity, sustained frequency ranges, abrupt changes in amplitude, or effects. Example effects may include, but are not limited to, warbling, oscillating, explosive, pulsing, compounding waveforms creating sum and difference output, constructive and destructive interference, and/or the like.

In some embodiments, the audio stimulus prediction machine learning model is configured to determine, for each audio stimulus sample, an effectiveness measure. In some embodiments, to generate the effectiveness measure for an audio stimulus sample, the audio stimulus prediction machine learning model processes feature data generated based at least in part on applying frequency spectrum analysis to each audio stimulus sample. In some embodiments, the input data to the audio stimulus prediction machine learning model may include one or more vectors describing feature data associated with an input audio stimulus sample, while output data of the audio stimulus prediction machine learning model may include an effectiveness measure for an input audio stimulus sample, where the noted effectiveness measure could be an atomic value or a vector.

Subsequent to step/operation 708, the example process 700 proceeds to step/operation 710. At step/operation 710, audio stimulus prediction computing entity 10 identifies one or more audio stimulus patterns of the subset of effective audio stimulus samples. In some embodiments, audio stimulus prediction computing entity 10 classifies sounds with like audio stimulus patterns (e.g., using an audio prediction machine learning model). In some embodiments, the audio stimulus prediction computing entity 10 may parse/classify sounds from an effective audio stimulus sample into a plurality of particular sections. By way of example, an audio stimulus sample may comprise a rain section, a thunder section, a lightning section, and a bird chirping section. In the above example, the audio stimulus prediction computing entity 10 may determine that the sequence of sounds associated with the effective audio stimulus sample triggers activity in a target area of the brain (e.g., the patient's frontal lobe/right hemisphere of the brain) as indicated by the patient's audio stimulus map. Based at least in part on further analysis of the effective audio stimulus sample, the audio stimulus prediction computing entity 10 may further determine that the bird chirping section is the effective portion of the audio stimulus sample (e.g., based at least in part on a spike in the waveform that is associated with the bird chirping section). Subsequently, the audio stimulus prediction computing entity 10 may identify audio stimulus patterns of the bird chirping section such as volume intensity, frequency range(s), changes in amplitude, warbling effect, and the like. For example, audio stimulus prediction computing entity 10 may identify sounds of other birds that contain similar peaks, rises, or falls, and/or spacing between the analyzed portions/sections of the sound.

In some embodiments, identifying one or more audio stimulus patterns of the subset of effective audio stimulus samples comprises providing additional/random sequences of audio stimulus samples associated with the effective subset of audio stimulus samples for further testing in order to identify the most stimulating sounds and associated parameters thereof. In some embodiments, by repeating this same process for all audio stimulus samples whose effectiveness measure satisfies a first patient response threshold (e.g., audio stimulus samples associated with EEG signals above a predetermined threshold value), additional samples that satisfy a second patient response threshold (e.g., a second patient response threshold that is greater than the first patient response threshold) may be identified. The audio stimulus prediction computing entity 10 may thus identify as effective certain broad sounds and/or more singular sounds that were associated with an optimal patient response threshold (e.g., peak EEG response). In some embodiments, serial sounds, sequential sounds, or combinations of sounds may be more effective than a single sound in isolation. In some embodiments, audio stimulus prediction computing entity 10 may identify stimulation occurring within a particular time period subsequent to exposure to sound(s) (e.g., within ten seconds, within a minute, and so on). In some examples, audio stimulus prediction computing entity 10 may further test/evaluate sounds that are played prior to a detected spike in a patient's response. In various examples, the identified sounds may include voices, music, specific notes of music, birds, and/or the like. Accordingly, based at least in part on machine learning techniques, the audio stimulus prediction computing entity 10 may identify sounds and/or audio patterns that may not be intelligible or identifiable by human ears.

Subsequent to step/operation 710, the example process 700 proceeds to step/operation 712. At step/operation 712, audio stimulus prediction computing entity 10 generates an audio treatment profile for the patient based at least in part on audio stimulus patterns of the effective subset of audio stimulus samples. The audio treatment profile may comprise a data object that describes audio treatment samples and/or raw audio data (e.g., comprising a plurality of sounds) associated therewith. In some embodiments, the audio treatment profile may comprise a document, machine-readable code, computer-executable instructions/parameters for generating and/or obtaining raw audio data. Each audio treatment sample may be defined by one or more audio stimulus patterns that are deemed effective for stimulating the patient such as duration, intensity, pitch, frequency, and/or the like. Additionally, each audio treatment sample may be associated with one or more parameters (e.g., environmental conditions) such as time of day, and/or physiological parameters. In some embodiments, the audio treatment samples and/or raw audio data may comprise a voice recording that is periodically played at one or more particular times of the day (e.g., on a loop, in response to certain triggers, predetermined conditions being met, and/or the like).

Subsequent to step/operation 712, the example process 700 proceeds to step/operation 714. At step/operation 714, audio stimulus prediction computing entity 10 performs one or more prediction-based tasks based at least in part on the audio treatment profile. As noted above, the one or more prediction-based tasks may comprise providing the audio treatment profile for use in conjunction with a patient monitoring device 40 and/or other electronic device(s) in order to provide audio stimulation to a patient. In some embodiments, performing the prediction-based task comprises transmitting the audio treatment profile to an audio stimulation device 20, where the audio stimulation device 20 may use the audio treatment profile to select/generate one or more audio recordings and present the selected/generated audio recordings to a patient during one or more audio recording sessions (where timing and/or other contextual parameters of the audio recording sessions may also be determined based at least in part on the audio treatment profile).

### Exemplary Techniques for Providing Audio Stimulation Treatments

Referring now to FIG. 6, a flowchart diagram illustrating an example process 600 performed by an audio stimulation device 20 and/or a patient monitoring device 40 in accordance with some embodiments of the present disclosure is provided. In some embodiments, the example audio stimulation device 20 and the example patient monitoring device 40 may be in electronic communication such that they can exchange information and/or data with one another.

Beginning at step/operation 602, the audio stimulation device 20 receives a plurality of audio stimulus samples. In various embodiments, the plurality of audio stimulus samples may be provided by an audio stimulus prediction computing entity 10 (e.g., obtained from one or more databases or sound libraries). In some embodiments, at least a portion of the plurality of audio stimulus samples may be provided (e.g., recorded, uploaded) and/or selected (e.g., from a plurality of candidate audio stimulus samples) by a clinician, therapist, or family member (e.g., interfacing with the audio stimulation device 20).

Subsequent to step/operation 602, the process 600 proceeds to step/operation 604. At step/operation 604, the audio stimulation device 20 provides (e.g., presents) the plurality of audio stimulus samples to a patient (e.g., a comatose patient). In various embodiments, the plurality of audio stimulus samples may be provided via an electronic device in electronic communication with the audio stimulation device 20 (e.g., headphones, a speaker, and/or the like), and/or the patient monitoring device 40. In some embodiments, the plurality of audio stimulus samples may include musical notes, generic open-source music compositions, and external environmental sounds associated with or familiar to the patient (e.g., traffic, animals, wind, rain, or the like). By way of example, the musical notes may comprise low, medium, and high scales using low, medium, and high instruments, percussive instruments in their own ranges, generic open-source music composition, and/or various excerpts of multiple musical genres. Additionally, audio stimulus samples may include sounds that are familiar to the patient such as favorite music or playlists (e.g., as selected by family members), recordings by individuals known to the patient (e.g., family, friends), sounds from familiar or historical activities (e.g., vacations, video recordings), the patient's own voice, or the like. In some embodiments, the audio stimulus samples may be selected based at least in part by analyzing a patients social media data, personal documents, and other sources to identify candidate sounds that are likely to be effective for the patient. By way of example, if a child is in a comatose state, a parent may grant permission for the child's social media feed to be evaluated for candidate sounds with a high likelihood of triggering a patient response. In yet another example, the audio stimulus samples may be determined based on a patient's occupation. For example, if a patient is a professional basketball referee, sounds that are associated with professional basketball games (e.g. a whistle blowing, a timeout buzzer, and the like) may be selected. Similarly, if the patient is a construction worker sounds that are associated with construction work (e.g. a truck backing up, jack hammers, traffic sounds, and the like) may be selected.

In some embodiments, variations to particular sounds may be provided for further analysis. Such variations may include left or right ear focus, variations in amplitude/volume of sounds (e.g., slow attack or bursts of sounds), time of day or the like. In various embodiments, each audio stimulus sample may be provided multiple times under different conditions and/or over an extended period of time (e.g., on multiple days covering a period of days or weeks).

In addition to providing the plurality of audio stimulus samples at step/operation 604, at step/operation 606, audio stimulation device 20 obtains patient response data associated with each of the plurality of audio stimulus samples (e.g., obtained via the patient monitoring device 40). For example, each time a particular audio stimulus sample is provided to a patient, data (e.g., patient response data such as brainwave information/data, physiological information/data, and/or the like) is generated based at least in part on patient response to the audio stimulus sample, where each data item may be tagged with the date/time) when the particular audio stimulus sample is provided and/or contextual parameters (e.g., environmental information/data such as ambient temperature, light level and/or the like) of an environment in which the particular audio stimulus sample is provided. In some embodiments, patient response data is obtained via the patient monitoring device 40, IoT device sensors, one or more computing entities within a predetermined range of the patient, and/or the like. As noted above, patient response data may comprise brainwave information/data (e.g., obtained via patient monitoring device electrodes), physical movement/muscle information/data, image data, physiological information/data, and/or the like. In some embodiments, additional parameters and/or variation information associated with each audio stimulus sample provided to the patient may be recorded/stored in conjunction with a set of patient response data. The results data from a plurality of test iterations may be stored within or in association with a patient profile, such that the results data from the plurality of test iterations may be aggregated and utilized to generate a cohesive report for the patient.

Subsequent to providing a plurality of audio stimulus samples at step/operation 604 and obtaining patient response data at step/operation 606, the example process 600 proceeds to step/operation 608. At step/operation 608, the audio stimulation device 20 (and/or in some examples, the patient monitoring device 40) provides an event data object (e.g., to an audio stimulus prediction computing entity 10). The event data object may be or comprise a data object storing and/or providing access to the plurality of audio stimulus samples and patient response data associated with the patient. The event data object may comprise sensor data describing the response of a patient when exposed to the plurality of audio stimulus samples. The event data object may describe one or more recorded events associated with the patient. In some embodiments, an event data object may comprise audio information/data, location information/data, image/video sensor information/data, physiological information/data, biometric information/data, environmental information/data, combinations thereof, and/or the like. In some embodiments, the event data object may comprise sensor data describing recorded patient response data (e.g., neural activity information/data, physiological information/data, image data, body temperature data, and/or the like) of the noted patient that is captured in conjunction with and/or responsive to delivery of the plurality of audio stimulus samples provided to the patient.

At step/operation 610, audio stimulation device 20 obtains (e.g., requests, receives, or the like) an audio treatment profile data object (e.g., from the audio stimulus prediction computing entity 10), where the audio treatment profile data object may be generated by the audio stimulus prediction computing entity 10 in accordance with the process 700 of FIG. 7. As noted above, in various examples, the audio treatment profile data object may comprise raw audio data, a document, or machine-readable code. The audio stimulation device 20 may, in certain embodiments, receive an applicable stored patient profile for a patient based at least in part on user input received via a user interface of the audio stimulation device 20 (or based at least in part on operator input data received from an audio stimulation device 20 associated with the patient monitoring device 40). It should be understood that an appropriate patient profile data object may be identified via any of a variety of alternative mechanisms, such as by identifying a patient profile associated with a particular audio stimulation device that is within communication range of the patient monitoring device 40. The audio stimulation device 20 may periodically request an updated audio treatment profile data object for the patient from the audio stimulus prediction computing entity 10. In some embodiments, the audio stimulation device 20 may generate at least a portion of data stored within the audio treatment profile data object. In one example, the audio stimulation device 20 may generate an initial audio treatment profile data object for a patient based at least in part on evaluation of user sensor data collected via one or more sensors of the patient monitoring device 40. In some embodiments, the audio stimulation device 20 may determine initial operating parameters and/or generate an audio treatment profile data object by monitoring the patient (e.g., obtaining and analyzing sensor data collected via one or more sensors of the patient monitoring device 40 for an initial time period). In some embodiments, the audio stimulation device 20 may provide (e.g., transmit, send) an event data object to the audio stimulus prediction computing entity 10 for generating and storing the audio treatment profile data object within a data storage area associated with the audio stimulus prediction computing entity 10. Subsequent to periodically receiving new information and/or data, the audio stimulus prediction computing entity 10 may update the audio treatment profile data object stored in conjunction with a patient profile data object and provide (e.g., transmit) an updated audio treatment profile data object periodically or on request.

Subsequent to obtaining the audio treatment profile data object at step/operation 610, the process 600 proceeds to step/operation 612. At step/operation 612, the audio stimulation device 20 provides audio treatment based at least in part on the audio treatment profile data object. In some embodiments, the audio stimulation device 20 may store the audio treatment profile and programmatically and/or automatically provide audio treatment samples (e.g., cause generation of sound waves) via a speaker or headphones operatively coupled thereto.

Additionally, the audio stimulation device 20 may coordinate the collection of new sensor data (e.g., brainwave information/data, physiological information/data) via the patient monitoring device 40 and/or one or more other electronic devices or sensors in conjunction while the patient is receiving audio treatment. For example, the audio stimulation device may record treatment sessions and collect EEG data in order to facilitate analysis of the patient's response to the audio treatment sessions.

In some embodiments, at least a portion of the audio treatment may be managed and/or coordinated by an operator (e.g., clinician or family member) that is interacting with the audio stimulation device 20 (e.g., via a user interface). In various examples, the audio stimulation device 20 may automatically and/or dynamically vary timeframes for audio treatment sessions and/or sequences of provided audio treatment samples. For instance, the audio stimulation device 20 may provide random sequences of stimulating sounds that were deemed effective in order to explore every possible combination and identify even more effective sequences. In some embodiments, sensor data collected by the audio stimulation device 20 and/or patient monitoring device 40 may be monitored for indications of positive changes to the audio treatment sessions such as increased brain activity and/or body movements as treatment progresses. In some embodiments, values, scores, reports, and/or graphical representations of trends in the patient response data may be provided for presentation to an operator/clinician (e.g., via a user interface). For instance, a change from 2% measured responsiveness to over 10% measured responsiveness over a period of time may be a positive therapy indication. In some embodiments, the audio stimulation device 20 may generate an alert in response to identifying positive therapy indications above or below a predetermined threshold (e.g., +/−10%). Optionally, in addition to and/or in conjunction with audio treatment sessions, a therapist or family member may provide additional stimulus (e.g., by applying reverse stimulus therapy to key areas of the body where increased activity has been triggered by audio treatment sessions). By way of example, if audio treatment sessions result in increased instances of finger twitching over time, audio stimulation device 20 may generate an alert/report indicating that physical therapy directed to the patient's hand (e.g., applying pressure, hand holding or the like) should be provided in conjunction with and/or addition to the audio treatment sessions. In some embodiments, the audio stimulation device 20 may monitor/identify supplementary therapies in sensor data (e.g., image/video sensor data) captured by an image sensor in the patient's environment. In some examples, data associated with successful audio treatment programs/sessions may be de-identified and cloud stored for use by other computing entities.

In an instance in which audio treatment sessions are not successful and/or lead to negative therapy indications or identifiable trends in the data being collected, the audio stimulation device 20 and/or audio stimulus prediction computing entity 10 may continue to dynamically vary and/or modify parameters of the audio treatment profile for the patient. For example, the audio stimulation device 20 may continually introduce randomly generated patterns of sound waves to further attempt to trigger an improved patient response. In some examples, in response to low levels of patient response to audio treatment sessions, an operator or clinician may choose to continue random therapy in order to locate the most minute brain function, as such patients with limited patient responses (and even those with minimal to no recorded brain activity) can recover.

### Exemplary Techniques for Updating an Audio Treatment Profile

Referring now to FIG. 8, a flowchart diagram illustrating an example process 800 for providing an updated audio treatment profile data object by an audio stimulus prediction computing entity 10 or another computing entity, in accordance with some embodiments of the present disclosure is provided.

Beginning at step/operation 802, the audio stimulus prediction computing entity 10 obtains a patient profile data object describing patient information/data. In some embodiments, the patient profile data object may be provided by a remote computing entity (e.g., a remote computing entity storing patient EMR data). The patient profile data object may describe various types of information associated with a particular patient including, but not limited to, age, gender, weight, height, body mass index (BMI), weight distribution and/or the like. In some embodiments, patient profile data objects describing patient information may be provided by one or more computing entities, one or more other wearable or health management, and/or the like. In some embodiments, step/operation 802 may be performed as part of registering a patient. For example, a patient profile data object for a patient may be generated/created as part of registration. However, as will be recognized, a patient profile may already exist and be stored in a patient profile database. In such a case, registration may link the patient to an existing patient profile. Each patient profile may be identifiable via one or more identifiers (e.g., social security numbers, patient IDs, member IDs, participant IDs, usernames, one or more globally unique identifiers (GUIDs), universally unique identifiers (UUIDs), and/or the like) that are configured to uniquely identify the patient profile. As part of registering a patient, audio stimulus prediction computing entity 10 may obtain (e.g., request and receive) various data objects describing information/data associated with a patient.

Subsequent to step/operation 802, the method 800 proceeds to step/operation 804. At step/operation 804, the audio stimulus prediction computing entity 10 stores an audio treatment profile data object in conjunction with the patient profile data object. As noted above, in various embodiments, audio stimulus prediction computing entity 10 receives one or more data objects describing the patient information/data for generation/creation of and/or storage in conjunction with a patient profile data object. In some embodiments, a patient's EMR may be associated with and/or otherwise stored in conjunction with the patient profile data object. The audio stimulus prediction computing entity 10 may store an event data object in conjunction with the patient profile data object.

Subsequent to step/operation 804, the method 800 proceeds to step/operation 806. At step/operation 806, the audio stimulus prediction computing entity 10 provides (e.g., transmits, sends and/or the like) the audio treatment profile data object to the audio stimulation device 20 and/or patient monitoring device 40 to facilitate operations.

Subsequent to step/operation 806, the method 800 proceeds to step/operation 808 At step/operation 808, audio stimulus prediction computing entity 10 periodically obtains an updated audio treatment profile data object describing patient information and/sensor data obtained by the controller of the patient monitoring device 40 and/or audio stimulation device 20 including, e.g., audio treatment data, patient response data, biometric data, and/or the like.

As noted, the audio treatment profile data object for a patient may be periodically updated (e.g., as new data is provided, as audio treatment sessions are provided over time, and/or the like). Accordingly, the audio stimulus prediction computing entity 10 and the audio stimulation device 20 may implement a feedback loop that updates the audio treatment profile data object for a patient based at least in part on an inferred determination regarding changes in patient responsiveness over time. For example, the audio stimulus prediction computing entity 10 may determine that a current audio treatment protocol is not improving patient responsiveness over time and therefore more testing needs to be performed to identify more effective sounds. Similarly, the audio stimulus prediction computing entity 10 may determine that a current audio treatment protocol is improving patient responsiveness over time and therefore additional sessions and/or supplementary therapies should be introduced.

Subsequent to step/operation 808, at step/operation 810, in response to obtaining/receiving an updated patient profile data object, the audio stimulus prediction computing entity 10 updates the audio treatment profile data object for the patient which is stored in conjunction with the patient profile data object. The audio stimulus prediction computing entity 10 may update the audio treatment profile data object based at least in part on new patient EMR data, biometric data and/or sensor data provided by other computing entities and/or the like. In so doing, the audio stimulus prediction computing entity 10 can refine the audio stimulus profile over time and provide more effective stimulation. Additionally, the most effective stimulation protocols for a particular patient, and particular population subgroups sharing certain audio stimulus patterns (e.g., target brain area, target body part, similar medical history/profile, age, gender and so on) can be identified over time. In certain embodiments, the audio stimulus prediction computing entity 10 may be configured to refine the audio stimulus profile for a patient using an audio stimulus prediction machine learning model (e.g., a trained neural network). Moreover, updated information based at least in part on new user features (e.g., patient response data, medical history including recent medical procedures and/or the like) can be provided for updating the audio treatment profile data object, which may be utilized to refine audio treatments to be utilized for certain population subgroups. In some embodiments, the audio stimulation device 20 and/or one or more other computing devices may be are configured to obtain (e.g., monitor, detect, and/or the like) additional body data and provide data object(s) associated therewith. The body data may be or comprise physiological information/data, biometric information/data, heart rate data, oxygen saturation data, pulse rate data, body temperature data, breath rate data, perspiration data, blood pressure data, neural activity data, cardiovascular data, pulmonary data, and/or various other types of information/data which may be relevant for updating the audio treatment profile data object storing the plurality of stimulation protocols for a patient.

Subsequent to updating the patient profile data object at step/operation 810, at step/operation 812, the audio stimulus prediction computing entity 10 transmits an updated audio treatment profile data object to the audio stimulation device 20 and/or patient monitoring device 40. In various embodiments, the audio stimulus prediction computing entity 10 and the audio stimulation device 20 periodically update and provide (e.g., send, transmit) audio treatment profile data objects and in so doing effectively incorporate real-time patient response data and patient profile information/data in a continuous feedback loop.

### Exemplary Techniques for Generating User Interface Data

In various embodiments, a variety of sources (e.g., audio stimulus prediction computing entity 10) may provide (e.g., transmit, send) a mobile application for download and execution on an audio stimulation device 20 (e.g., in response to a request to download the mobile application generated at the audio stimulation device 20). In another embodiment, the mobile application may be pre-installed on the audio stimulation device 20. And in yet another embodiment, the mobile application may be a browser executing on the audio stimulation device 20. The mobile application may comprise computer-executable program code (e.g., a software application) that provides the functionality described herein. The mobile application may enable various functionalities as discussed herein. Moreover, although specifically referenced as a mobile application, it should be understood that the mobile application may be executable by any of a variety of computing entity types, such as desktop computers, laptop computers, mobile devices, and/or the like. In various embodiments, instructions may be automatically generated (e.g., by the audio stimulus prediction computing entity 10) or provided based at least in part in response to clinician input/instructions provided by a clinician interacting with the audio stimulus prediction computing entity 10. The instructions may comprise messages in the form of banners, headers, notifications, and/or the like.

In some embodiments, at least a portion of the obtained patient monitoring device sensor data may be transferred to the audio stimulation device 20 and/or the audio stimulus prediction computing entity 10 for performing at least a portion of the required operations. The patient monitoring device 40 or audio stimulation device 20 may be configured to provide information/data in response to requests/queries received from the audio stimulus prediction computing entity 10. In various embodiments, the patient monitoring device 40 may be managed, calibrated and/or otherwise controlled at least in part by the audio stimulus prediction computing entity 10. The audio stimulus prediction computing entity 10 may generate a user interface data object based at least in part on a patient profile data object and provide (e.g., transmit, send) the patient interface data object to one or more client computing entities.

In some embodiments, an operator/clinician may generate and provide user interface data for presentation via a user interface depicting audio treatment information/data including brainwave information/data obtained during audio treatment sessions. In some embodiments, the operator or clinician may select a target area of the brain (e.g., associated with a particular injury type) for the audio treatment profile to be generated by the audio stimulus prediction computing entity 10. In some examples, the audio stimulus prediction computing entity 10 may identify key sounds that correlate with target areas of the brain and/or electrode nodes of the brain. In some embodiments, the operator/clinician may select audio stimulus samples (e.g., in particular ranges and/or having particular audio stimulus patterns) for the audio stimulus prediction computing entity 10. By way of example, an operator may select a frequency range between 2000 and 3000 Hz. In another example, the operator may select a frequency range associated with a brainwave type (e.g., beta, alpha, theta, or delta brainwaves). Using stored data and machine learning techniques, the audio stimulus prediction computing entity 10 may identify input data, including personal input data falling within selected spectral regions and utilize the identified data to generate the audio stimulus profile (and an audio therapy schedule) for the patient.

As noted above, the audio stimulus prediction computing entity 10 may deem certain audio stimulus samples effective based at least in part on patient response data (e.g., brainwave information/data and/or voluntary/involuntary muscular activity data captured via an image sensor, motion sensor, inductive probe(s), and/or the like). In some embodiments, the clinician may select the brain area(s) and/or body area(s) deemed responsive to specific forms of audio stimulus based at least in part on data provided by the audio stimulus prediction computing entity 10. Additionally, the clinician may make modifications to an audio stimulus profile by selecting target brain area(s) and/or target body area(s), a target medical response and/or by increasing delivery parameters (e.g., loudness, frequency, or the like). By way of example, a target medical response for a patient that is experiencing slow heart rhythms may be to increase the heart rate of the patient. Accordingly, the audio stimulus prediction computing entity 10 may provide audio treatment samples that have been determined to increase the patient's heart rate or heart rates of similar patients. In certain embodiments, an example audio stimulus profile (e.g., raw audio data) may simulate the patient's previous daily life in a cyclical manner so as to approximate the experience of day to day activities. For example, audio stimulus samples may simulate periods of wakefulness or sleep and may further target particular behaviors of feelings. In one example, if a patient's EHR indicates that he or she is struggling with anger or nervousness, then the audio stimulus treatment for the patient may include sounds to reduce Beta brainwaves which are associated with feelings of anger and nervousness.

FIG. 9 provides an operational example 900 of a user interface 901 that is generated based at least in part on dynamically updating user interface data, where the patient interface data may be generated based at least in part on an audio stimulus profile for a patient. In various embodiments, the audio stimulation device 20 and/or client computing entity generates user interface data (e.g., one or more data objects) which is provided (e.g., transmitted, sent and/or the like) for presentation by the user interface 901 of an audio stimulation device 20 and/or client computing entity. The user interface 901 may comprise various features and functionality for accessing, and/or viewing data objects and/or alerts. The user interface 901 may also comprise messages in the form of banners, headers, notifications, and/or the like. As depicted in FIG. 9, the patient interface data comprises an indication of patient information 902, patient profile information 904 and audio treatment information 906 associated with a patient. Additionally, the patient interface data includes user selectable objects 903A, 903B, 903C and 903D to facilitate user/clinician interaction with the system and modification of operational parameters and settings.

As will be recognized, a variety of other approaches and techniques can be used to adapt to various needs and circumstances. The present disclosure provides systems that utilize machine learning techniques to identify optimal treatments, e.g., key ranges of sound frequencies for comatose patients that may result in reawakening and/or shortening patient awakening timeframes. Additionally, learned data can be recommended by the system in relation to specific injuries, conditions, and successes to automatically shape input sound data to conform to the specifications. In some embodiments, a cloud storage system of de-identified data is provided. Specific successful/unsuccessful treatments and data in relation to age, ethnicity, geographical or environmental considerations can be provided. Additionally, relational parameters such as general health data, injury types, biometric data and demographics can be processed and over time present common machine-learning clustered treatment programs for specific conditions. In certain embodiments, this can be used and proven in medically induced coma situations to awaken patients more quickly or treat those that do not awaken in the intended manner. The system will apply known parameters of success to generate a treatment plan that over time can generate more precise and successful physician instructions. Biomedical responses to therapy can be learned and valuable to the physician. By way of example, the system may detect increase in urine output measurement associated with an audio treatment profile and thus facilitate treatment of a dehydrated comatose patient. In another example, as described herein, learned responses to therapy that may increase heart rate can be valuable to the physician to treat low heart rate in comatose patients. Learned medical data in this perpetual and automated study can be of insurmountable value to future treatments of these patients for many conditions. Faster treatment may be possible especially considering instances where time is of the essence. With cumulative statistics, this system can be used to estimate projected costs and also be used to assist as data consideration for determination of continued life-support.

Accordingly, as described above, the apparatuses, systems, and methods described herein provide a robust audio stimulation system. Moreover, various embodiments of the present disclosure provide audio stimulus prediction machine learning models that can make inferences based at least in part on sensory data in order to provide more effective stimulation compared to the state-of-the-art systems. Accordingly, various embodiments of the present disclosure make substantial technical contributions to the field of monitoring devices for comatose patients and substantially improve state-of-the-art systems.

Accordingly, as described above, various embodiments of the present invention provide practical applications by improving therapeutic stimulation of comatose (or partially comatose) patients with greater effectiveness and efficiency. For example, various embodiments of the present invention generate audio recordings to present to comatose (or partially comatose) patients based at least in part on audio stimulus patterns from those audio stimulus samples that are deemed to be more effective in inducing patient response from comatose (or partially comatose) patients. In doing so, various embodiments of the present improve the effectiveness and efficiency of stimulating comatose (or partially comatose) patients and provide practical solutions for enabling therapeutic stimulation of comatose (or partially comatose) patients.

### VI. CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only, and not for purposes of limitation.

1. A computer-implemented method for generating an audio treatment profile for a patient, the computer-implemented method comprising:
retrieving, by one or more processors, a plurality of audio stimulus samples;
receiving, by the one or more processors, an event data object comprising sensor data describing patient response data of the patient when exposed to the plurality of audio stimulus samples;
generating, by the one or more processors and based at least in part on the plurality of audio stimulus samples and the event data object, an audio stimulus map for the patient, wherein the audio stimulus map comprises a mapping of each of the plurality of audio stimulus samples to the patient response data;
determining, by the one or more processors, based at least in part on the audio stimulus map and using an audio stimulus prediction machine learning model, an effective subset of the plurality of audio stimulus samples, wherein each audio stimulus sample in the effective subset is associated with a patient response measure that satisfies a patient response measure threshold;
identifying, by the one or more processors, one or more audio stimulus patterns of the effective subset; and
generating, by the one or more processors, the audio treatment profile based at least in part on the one or more identified audio stimulus patterns of the subset of effective audio stimulus samples, wherein the audio treatment profile may be used to present one or more audio recordings to the patient.

2. The computer-implemented of claim 1, wherein the audio stimulus prediction machine learning model further comprises:
a first sub-model configured to generate the audio stimulus map; and
a second sub-model configured to generate the audio treatment profile.

3. The computer-implemented method of claim 1, wherein the effective subset is determined based at least in part on a filtering technique or a destructive interference technique.

4. The computer-implemented method of claim 1, wherein the one or more identified audio stimulus patterns comprise one or more of a time of day pattern for the effective subset, an ear focus pattern for the effective subset, and audio patterns for the effective subset.

5. The computer-implemented of claim 1, wherein:
each audio stimulus sample in the effective subset is associated with one or more of a target location of the patient's brain, a target muscle group, and a measurable physical response, and
the sensor data comprises one or more of neural activity data, heart rate data, body temperature data, and cardiovascular data.

6. The computer-implemented method of claim 1, further comprising:
providing, by the one or more processors, an audio treatment profile data object describing the audio treatment profile to a patient monitoring device configured to provide audio stimulation and physical stimulation to the patient; and
storing, by the one or more processors, information associated with the audio treatment profile to a patient profile.

7. The computer-implemented method of claim 1, wherein the plurality of audio stimulus samples are associated with one or more of the patient's social media data and personal documents.

8. The computer-implemented method of claim 1, further comprising:
dynamically adjusting, by the one or more processors, the audio treatment profile for the patient based at least in part on patient response data associated with the audio treatment profile.

9. An apparatus for generating an audio treatment profile for a patient, the apparatus comprising at least one processor and at least one memory including program code, the at least one memory and the program code configured to, with the at least one processor, cause the apparatus to at least:
retrieve a plurality of audio stimulus samples;
receive an event data object comprising sensor data describing patient response data of the patient when exposed to the plurality of audio stimulus samples;
generate, based at least in part on the plurality of audio stimulus samples and the event data object, an audio stimulus map for the patient, wherein the audio stimulus map comprises a mapping of each of the plurality of audio stimulus samples to the patient response data;
determine, based at least in part on the audio stimulus map and using an audio stimulus prediction machine learning model, an effective subset of the plurality of audio stimulus samples, wherein each audio stimulus sample in the effective subset is associated with a patient response measure that satisfies a patient response measure threshold;
identify one or more audio stimulus patterns of the effective subset; and
generate the audio treatment profile based at least in part on the one or more identified audio stimulus patterns of the subset of effective audio stimulus samples, wherein the audio treatment profile may be used to present one or more audio recordings to the patient.

10. The apparatus of claim 9, wherein the audio stimulus prediction machine learning model further comprises:
a first sub-model configured to generate the audio stimulus map; and
a second sub-model configured to generate the audio treatment profile.

11. The apparatus of claim 9, wherein the subset of effective audio stimulus samples is determined based at least in part on a filtering technique or destructive interference technique.

12. The apparatus of claim 9, wherein the one or more identified audio stimulus patterns comprise one or more of a time of day pattern for the effective subset, an ear focus pattern for the effective subset, and audio patterns for the effective subset.

13. The apparatus of claim 9, wherein:
each audio stimulus sample in the effective subset is associated with one or more of a target location of the patient's brain, a target muscle group, and a measurable physical response, and
the sensor data comprises one or more of neural activity data, heart rate data, body temperature data, and cardiovascular data.

14. The apparatus of claim 9, wherein the at least one memory and the program code are configured to, with the at least one processor, cause the apparatus to at least:
provide, by the one or more processors an audio treatment profile data object describing the audio treatment profile to a patient monitoring device configured to provide audio stimulation and physical stimulation to the patient; and
store, by the one or more processors, information associated with the audio treatment profile to a patient profile.

15. The apparatus of claim 9, wherein the plurality of audio stimulus samples are associated with one or more of the patient's social media data and personal documents.

16. The apparatus of claim 9, wherein the at least one memory and the program code are configured to, with the at least one processor, cause the apparatus to at least:
dynamically adjust, by the one or more processors, the audio treatment profile for the patient based at least in part on patient response data associated with the audio treatment profile.

17. A computer program product for determining an audio treatment profile with respect to an event data object, the computer program product comprising at least one non-transitory computer-readable storage medium having computer-readable program code portions stored therein, the computer-readable program code portions configured to:
retrieve a plurality of audio stimulus samples;
receive an event data object comprising sensor data describing patient response data of the patient when exposed to the plurality of audio stimulus samples;
generate, based at least in part on the plurality of audio stimulus samples and the event data object, an audio stimulus map for the patient, wherein the audio stimulus map comprises a mapping of each of the plurality of audio stimulus samples to the patient response data;
determine, based at least in part on the audio stimulus map and using an audio stimulus prediction machine learning model, an effective subset of the plurality of audio stimulus samples, wherein each audio stimulus sample in the effective subset is associated with a patient response measure that satisfies a patient response measure threshold;
identify one or more audio stimulus patterns of the effective subset; and
generate the audio treatment profile based at least in part on the one or more identified audio stimulus patterns of the subset of effective audio stimulus samples, wherein the audio treatment profile may be used to present one or more audio recordings to the patient.

18. The computer program product of claim 17, wherein the audio stimulus prediction machine learning model further comprises:
a first sub-model configured to generate the audio stimulus map; and
a second sub-model configured to generate the audio treatment profile.

19. The computer program product of claim 17, wherein the subset of effective audio stimulus samples is determined based at least in part on a filtering technique or destructive interference technique.

20. The computer program product of claim 17, wherein the one or more identified audio stimulus patterns comprise one or more a time of day pattern for the effective subset, an ear focus pattern for the effective subset, and audio patterns for the effective subset.
